(19) United States
(12) Patent Application Publication
(10) Pub. No.: US 2026/0306066 A1
(43) Pub. Date: Oct. 8, 2026

(54) METHOD AND SYSTEM FOR ULTRASONIC CHARACTERISATION OF A MEDIUM

(71) Applicants: CENTRE NATIONAL DE LA RECHERCHE SCIENTIFIQUE, PARIS (FR); ECOLE SUPÉRIEURE DE PHYSIQUE ET DE CHIMIE INDUSTRIELLES DE LA VILLE DE PARIS, PARIS (FR)

(72) Inventors: Flavien BUREAU, PARIS (FR); Elsa GIRAUDAT, BULLION (FR); Arthur LE BER, VIBEUF (FR); William LAMBERT, YVETOT (FR); Mathias FINK, MEUDON (FR); Alexandre AUBRY, IVRY SUR SEINE (FR)

(73) Assignees: CENTRE NATIONAL DE LA RECHERCHE SCIENTIFIQUE, PARIS (FR); ECOLE SUPÉRIEURE DE PHYSIQUE ET DE CHIMIE INDUSTRIELLES DE LA VILLE DE PARIS, PARIS (FR)

(21) Appl. No.: 19/494,636

(22) PCT Filed: Jun. 13, 2024
(86) PCT No.: PCT/EP2024/066388

§ 371 Date: Dec. 18, 2025

(30) Foreign Application Priority Data
Jun. 20, 2023 (FR) FR2306343

(51) Int. Cl. A61B 8/00; A61B 8/08; G01S 7/52

(52) U.S. Cl. CPC A61B 8/5223; A61B 8/4483; A61B 8/461; A61B 8/485; G01S 7/52057; A61B 8/08

(57) ABSTRACT

Method for ultrasonic characterisation of a medium including a step of generating a series of incident ultrasonic waves, a step of generating an experimental reflection matrix Rᵤᵢ(t) defined between the transmission basis (i) at the input and a reception basis (u) at the output, a step of determining a de-scanned reflection matrix R′ of the medium between an input virtual transducer computed from an input focusing of the experimental reflection matrix and an output virtual transducer computed from an output focusing of the experimental reflection matrix, these virtual transducers being located at the same depth. The method further includes a step of extracting an optimal position offset or an optimal sound speed model from this focal spot image.

### FIELD

The present description relates to methods and systems for ultrasonic characterisation of a medium, and applies in particular to medical imaging as well as to non-destructive testing of a medium, and more generally to any field that may use a multi-element transducer array to measure reflection from a scattering medium.

### BACKGROUND

### Limitations of Ultrasound Imaging

In the field of ultrasound imaging, the aim is to construct an image of the reflectivity of a medium from the echoes backscattered by heterogeneities in the medium. This is the principle of the ultrasound scanner used in medical imaging, which is used in particular to view a person's internal anatomy. Usually, in order to construct an ultrasound image, the medium is assumed to be homogeneous, with a constant speed of sound c₀. However, the assumption of a homogeneous medium is not valid in most practical cases, particularly in the imaging of parts of human or animal bodies. For example, in the case of ultrasounds of the liver, the acoustic waves pass through a succession of layers of fat and muscle before reaching the target organ. In this case, the speed of sound may vary, for instance, between 1450 m/s for the adipose tissue and 1600 m/s for the liver. The discrepancy between the actual distribution of the speed of sound within the medium and the velocity model used to construct the ultrasound image can lead to significant aberrations in the resulting image, potentially compromising, for example, a clinician's diagnosis during a medical examination.

As illustrated in FIG. 1, conventional ultrasound scanning methods use an array of piezoelectric transducers that can transmit and/or receive ultrasonic signals independently, each transducer being located at a position u in the bar supporting said array. The transducer array, placed facing the medium, allows the medium to be insonified and imaged in various ways. The conventional method consists in insonifying the medium using transmissions focused by a technique known as beamforming. As shown in FIG. 1(a), this method consists in applying a set of appropriate delays τ(uᵢₙ, xᵢₙ, z, c₀) based on a homogeneous sound speed model c₀ to the signals transmitted by each transducer, in order to cause the wavelets produced by each transducer to interfere at the targeted focal point at spatial position (xᵢₙ, z). Due to the physical limits of diffraction, the ultrasounds are transmitted through the aperture of the ultrasound probe and concentrated in a region often referred to as the “focal spot”, of lateral width dx.

To allow an ultrasound image to then be constructed, if necessary, a digital focusing step is also carried out on reception. The echoes captured by the transducers of the array, as shown diagrammatically in FIG. 1(b), are rephased by time-shifting. The delays τ(uₒᵤₜ, xₒᵤₜ, z, c₀) are identical to those applied on transmission, with the variable uₒᵤₜ denoting the position of each transducer. In the transmission phase, all signals interfere at the point of position (xᵢₙ, z) at the ballistic time t=z/c₀ if the sound speed model c₀ corresponds to reality. On reception, the signals originating from the same point (xₒᵤₜ=xᵢₙ) interfere by summation of the signals at the echo time t=2z/c₀. This summation yields the final result of the reception focusing. This confocal method with double focusing on transmission and reception, illustrated in FIG. 1, allows the reflectivity of the medium to be imaged directly with a lateral resolution dx and good contrast. However, this method is time-consuming, since it requires physical focusing on transmission at each point of the medium, or at least at a given depth, along each line of the constructed image representing the medium. Moreover, this image is often of low quality when the medium is heterogeneous, particularly when the speed of sound is not constant across the various regions constituting the observed medium. FIGS. 1(c) and (d) show a medium comprising an aberrating layer with a speed of sound different from that observed in the remainder of the medium. This results in an aberration of the acoustic wavefront, leading to distortion of the resulting ultrasound image and therefore to a deterioration in its resolution and contrast which is particularly problematic during a medical examination.

### Matrix Approach to Ultrasound Imaging

To compensate for these aberration problems, a matrix approach to ultrasound imaging has been developed in recent years. This approach is based on measuring an experimental reflection matrix of the medium under study, using the ultrasound probe.

One way of measuring this experimental reflection matrix is to successively transmit an ultrasonic pulse from each transducer in the array, whose position is identified by the coordinate uᵢₙ, as schematically illustrated in FIG. 2(a). This produces a divergent cylindrical (or spherical) incident wave. This wave is reflected by the scatterers in the medium, and the backscattered field is recorded by each transducer as a function of time, as shown in FIG. 2(b). By repeating this operation with each transducer used successively as a source, the experimental reflection matrix Rᵤᵤ(t) is determined, expressed in the transducer basis and consisting of the set of impulse responses R(uₒᵤₜ, uᵢₙ, t) between each pair of transducers. This matrix therefore contains a large quantity of information on the medium under study. However, the method assumes that the medium remains fixed throughout the duration of the measurements. In addition, the recorded signals have a poor signal-to-noise ratio because the medium is insonified by a single transducer.

A second way of constructing this experimental reflection matrix consists in insonifying the medium with a basis consisting of a series of plane waves. This method allows the problems mentioned above to be overcome. FIG. 2(c) illustrates the principle of this plane-wave illumination. A delay law τ′ is applied to each signal on transmission in order to form a wavefront inclined by an angle θᵢₙ with respect to the transducer array. On reception, illustrated in FIG. 2(d), the field backscattered by the medium, R(uₒᵤₜ, θᵢₙ, t), is measured by all the sensors at positions uₒᵤₜ for each incident plane wave. All of these responses together form an experimental reflection matrix Rue (t). The double focusing method described earlier can then be implemented numerically by time-shifting the measured signals before summing them coherently. This method gave rise to ultrafast imaging and elastography, and is described, for example, in the following document:

“Coherent plane-wave compounding for very high frame rate ultrasonography and transient elastography”, G. Montaldo et al. (IEEE Trans. Ultrason., Ferroelect. Freq. Control 56 489-506, 2009).

A third way to create this experimental reflection matrix consists in insonifying the medium using a basis consisting of divergent waves, as shown in FIGS. 2(e) and 2(f), which allows the acoustic field to be illuminated more broadly than when using plane waves. This technique, used in particular for super-resolution imaging, is described in the following document:

“Ultrafast imaging of the heart using Circular Wave Synthetic Imaging with Phased Arrays”, Couade et al., IEEE International Ultrasonics Symposium (2009).

Conventional imaging therefore consists of double focusing on transmission and reception at the same focal point for each pixel of the image (xᵢₙ=xₒᵤₜ), as shown in FIG. 3(a).

Matrix imaging, on the other hand, consists in separating the focal points on transmission and reception for the same echo time t, as shown in FIG. 3(b). This allows the determination of a focused reflection matrix Rᵣᵣ(t, c₀) consisting of the responses between input virtual transducers and output virtual transducers of respective spatial positions rᵢₙ=(xᵢₙ, zᵢₙ) and rₒᵤₜ=(xₒᵤₜ, zₒᵤₜ) corresponding to focal spots synthesised in input and output at different spatial positions, with depths determined by the echo time t. This matrix is obtained by beamforming from the experimental reflection matrix Rᵤᵢ(t). It provides considerably more information than the confocal image of conventional imaging. The focused reflection matrix can be synthesised in the time domain by delay laws or in the frequency domain by applying appropriate phase shifts.

Such matrix imaging was described in:
“Reflection Matrix Approach for Quantitative Imaging of Scattering Media”, William Lambert, et al., Phys. Rev. X 10, 021048, (2020), and subsequently in:
“Distortion matrix approach for ultrasound imaging of random scattering media”, W Lambert, et al.—Proceedings of the National Academy of Sciences, (2020).

It should be noted that, in these publications, the focused reflection matrix Rᵣᵣ(t, c₀) was considered between virtual transducers at the expected ballistic depth: zᵢₙ=zₒᵤₜ=zₜ=c₀t/2, where zₜ is the depth of the isochronous volume expected for a speed of sound c₀, and the isochronous volume corresponds to the set of points in the medium contributing to the signals received at the echo time t. This subset of the matrix Rᵣᵣ(t, c₀) is denoted Rₓₓ(zₜ, t c₀). While the diagonal coefficients (xₒᵤₜ=xᵢₙ) of this matrix Rₓₓ(zₜ, t, c₀) yield the synthetic confocal image at the depth zₜ, its off-diagonal coefficients provide information on potential aberration and multiple-scattering problems that may degrade the quality of this image.

The focused reflection matrix at the ballistic depth, Rₓₓ(zₜ, t, c₀), plays a pivotal role in ultrasound matrix imaging. It was initially studied to investigate the transverse evolution of the input and output focal spots, and then to compensate for aberrations by determining the optimal delay laws (or phase shifts) to be applied at the input and output of the reflection matrix. However, this approach is limited because it only allows aberrations to be probed transversely and does not make it possible to “relocate the scatterers to their true depth.” Consequently, any discrepancy between the sound speed model c₀ and the actual distribution c(r) leads to incorrect axial positioning of the imaged scatterers, as shown in FIG. 1.

In particular, as mentioned in patent applications US 2022/082693, US 2022/084496 and US 2022/082527, the focused reflection matrix was studied by the applicant beyond the ballistic depth, by scanning in depth the relative position of the virtual transducers. For a constant echo time t (i.e. for a fixed isochronous volume), the input (or output) focal spot of the device can be probed locally by scanning the spatial position rₒᵤₜ (or rᵢₙ) of the virtual transducer on output (or input). A defocus Δz=zₒᵤₜ−zᵢₙ can thus be measured at each spatial position rᵢₙ of the image, and an integrated sound speed map between the probe and each point rᵢₙ can be derived from this measurement.

However, this approach is not optimal in some cases because the virtual transducer at spatial position rₒᵤₜ (or rᵢₙ) is also affected by aberrations, which biases the estimator of the input (or output) focal spot.

### SUMMARY

The present description relates, according to a first aspect, to a method for characterising a medium by ultrasound, the method comprising:
a step of generating a series of incident ultrasonic waves USᵢₙ in a region of said medium, by means of an array of transducers, the series of incident ultrasonic waves forming a transmission basis i; and
a step of generating an experimental reflection matrix Rᵤᵢ(t) defined between the transmission basis i at the input and a reception basis u at the output, the coefficients of this matrix corresponding to the signals received by the transducers induced by the reflected ultrasonic waves.

The method further comprises:
a step of determining a de-scanned reflection matrix R′,
which comprises, for a set of points of the medium of abscissa x and echo time t,
responses of the medium calculated by beamforming from the experimental reflection matrix Rᵤᵢ(t), between an input virtual transducer at spatial position rᵢₙ=(x, zₜ+Δz) and an output virtual transducer at spatial position rₒᵤₜ=(x+Δx, zₜ+Δz), both virtual transducers being located at the same depth zₜ+Δz, where zₜ is the depth of the isochronous volume expected for a speed of sound c₀, and the isochronous volume corresponds to the set of points in the medium contributing to the signals received at the echo time t,
the responses of the de-scanned reflection matrix R′ being determined for a set of lateral offset values Δx including at least Δx=0 and for a set of depth offset values Δz,
said de-scanned reflection matrix R′ being formed such that each row corresponds to an element of a pair Δr={Δx, Δz} which corresponds to a focal spot image, and each column corresponds to an element of a pair {x, t}, which is written as:

$${R}^{′} = [ {R}^{′} ( \{ Δ x , Δ 𝓏 \} , \{ x , t \} ) ]$$

an extraction step in which, from each focal spot image, an optimal position offset Δrₒₚₜ, is determined, the optimal position offset Δrₒₚₜ={Δxₒₚₜ, ΔZₒₚₜ} being close to the maximum in said focal spot image.

Thanks to these arrangements, the method advantageously enables the medium to be probed locally at any point and in any direction with respect to the ballistic propagation time of the ultrasonic wave in the medium.

This method provides access to a large amount of local information on the medium, making it possible to characterise the ultrasonic wave focusing process locally and thus estimate the focal depth error Δzₒₚₜ.

This estimate is then very useful for improving the focusing and quality of the ultrasound image. This optimisation of ultrasound images is performed computationally, without requiring further transmissions and/or acquisitions, which is a significant advantage, especially for in vivo measurements. The optimisations can be carried out in real time or at a later stage, potentially on a different system and optionally on a remote one.

In various embodiments of the method, one and/or the other of the following arrangements may possibly be used.

According to one variant, the method further comprises:
a step of forming an ultrasound image by computing the reflectivity at a plurality of points in the medium, from the de-scanned reflection matrix R′, the reflectivity of each of these points in the medium corresponding to the response of the medium between the input virtual transducer at spatial position rᵢₙ=(x, zₜ+Δzₒₚₜ) and the output virtual transducer at spatial position rₒᵤₜ=r+Δrₒₚₜ=(x+Δxₒₚₜ, zₜ+ΔZₒₚₜ), said reflectivity being defined by:

$${ℐ}_{1} ( x , t ) = \vert {R}^{′} ( Δ r = Δ {r}_{opt} ( x , t ) , \{ x , t \} ) \vert .$$

According to one variant, the ultrasound image is transformed in the depth dimension using the sound speed model c₀ by:

$${\tilde{ℐ}}_{1} ( x , {𝓏}_{t} ) = {ℐ}_{1} ( x , t = 2 {𝓏}_{t} / {c}_{0} ) ,$$

in which zₜ is the depth of a pixel of the ultrasound image, or the depth of the isochronous volume.

According to one variant, the ultrasound image is displayed on a display device.

According to one variant, the method further comprises, prior to the extraction step:
a step of spatio-temporal averaging of the de-scanned reflection matrix R′ in which the values of the de-scanned reflection matrix R′ are averaged locally, along the lateral dimension x and along the echo time dimension t, for each element of the pair Δr={Δx, Δz}, the focal spot images then being averaged.

According to one variant:
in the spatio-temporal averaging step, an incoherent point spread function PSFᵢₙₑ is computed by the following formula:

$${PSF}_{inc} ( Δ r , \{ x , t \} ) = \sqrt{{〈 {\vert {R}^{′} ( Δ r , \{ {x}^{′} , {t}^{′} \} ) W ( {x}^{′} - x , {t}^{′} - t ) \vert}^{2} 〉}_{[ {x}^{′} , {t}^{′} ]}}$$

for a set of lateral offset values Δx including at least Δx=0 and for a set of depth offset values Δz, denoted as the pair Δr={Δx, Δz}, in order to obtain the focal spot image for the lateral position x and the echo time t,
in which:
<.> is an averaging operator with respect to the parameters of the pair {x′, t′}
R′ is the de-scanned reflection matrix,
W(x′, t′) is a spatio-temporal weighting window using the same parameters, and
in the extraction step, a maximum point of the focal spot image is sought, corresponding to a maximum of the magnitude of the points of the focal spot image, and the optimal position offset Δrₒₚₜ is the position of said maximum point in said focal spot image.

According to one variant:
in the spatio-temporal averaging step, a singular value decomposition (SVD) of a local focal spot matrix R^{(l)} is computed, said local focal spot matrix being derived from the de-scanned reflection matrix R′ by:

$${R}^{( l )} ( \{ x , t \} ) = [ {R}^{′} ( Δ r , \{ {x}^{′} , {t}^{′} \} ) W ( {x}^{′} - x , {t}^{′} - t ) \vert$$

in which:
R′ is the de-scanned reflection matrix,
W(x′, t′) is a spatio-temporal weighting window using the same parameters.
   The singular value decomposition (SVD) of said local matrix is written as:

$${R}^{( l )} ( x , t ) = {\sum }_{p} {λ}_{p} {U}_{p} ( \{ x , t \} ) {V}_{p}^{†} ( \{ x , t \} )$$

in which:
λₚ are the singular values of said local matrix,
Uₚ are the output eigenvectors,
Vₚ are the input eigenvectors,
a coherent point spread function PSF_{coh} is computed by the following formula:

$${PSF}_{coh} ( Δ r , \{ x , t \} ) = {U}_{1} ( Δ r , \{ x , t \} )$$

for a set of lateral offset values Δx including at least Δx=0 and for a set of depth offset values Δz, denoted as the pair Δr={Δx, Δz}, in order to obtain the focal spot image for the lateral position x and the echo time t, and
in the extraction step, a maximum point of the focal spot image is sought, corresponding to:
a maximum of the magnitude of the points of the focal spot image, or
a maximum of the magnitude of the derivative along the depth direction of the points of the focal spot image, or
a maximum of the magnitude of the derivative along the depth direction of the imaginary part of the points of the focal spot image, and
the optimal position offset Δrₒₚₜ is the position of said maximum point in said focal spot image.

According to one variant, in the extraction step, the lateral offset Δxₒₚₜ is considered to be zero, Δxₒₚₜ=0.

According to one variant, in the step of determining a de-scanned reflection matrix R′, the de-scanned reflection matrix R′ is formed only for lateral offset values Δx equal to zero, Δx=0.

According to one variant, in the spatio-temporal averaging step, the values of the de-scanned reflection matrix R′ are averaged along the lateral dimension x and the echo time t, only for focal spot images corresponding to a zero lateral offset, Δx=0.

According to one variant, in the extraction step, the optimal depth offset ΔZₒₚₜ is determined from the points of the focal spot image having a lateral offset Δx equal to zero, Δx=0.

According to one variant, an integrated sound speed c̅ₒₚₜ is determined from the optimal depth offset ΔZₒₚₜ and the optimal position offset Δrₒₚₜ={Δxₒₚₜ, ΔZₒₚₜ}, by:

$${c}_{opt} ( x , t ) = \frac{{c}_{0}}{\sqrt{1 + \frac{2 Δ {𝓏}_{opt} ( x , t )}{{c}_{o} t}}} ,$$

this integrated sound speed corresponding to an average sound speed between the transducer array and the depth of the isochronous volume.

According to one variant:
local sound speeds c(r) are calculated, for a set of pairs {x, t}, from integrated sound speed values c̅opt obtained for the focal spot images of said pairs {x, t},
According to one variant, the method further comprises:
a step of forming a local sound speed image of the medium from said set of local sound speed values.

According to one variant:
a de-scanned reflection matrix R′ is recalculated using the local sound speed values.

According to one variant, the beamforming calculations between the input virtual transducer and the output virtual transducer are performed based on the experimental reflection matrix Rᵤᵢ(t), using a forward delay of the ultrasonic waves between the transmission basis (i) and the input virtual transducer, and using a return delay of the ultrasonic waves between the output virtual transducer and the transducers of the reception basis (u).

According to one variant, the de-scanned reflection matrix R′ is calculated from the experimental reflection matrix Rᵤᵢ(t) by the following formula:

$${R}^{′} ( \{ Δ x , Δ z \} , \{ x , t \} , {c}_{0} ) = \frac{1}{{N}_{in} {N}_{out}} {\sum }_{{i}_{in} {u}_{out}} {R}_{ui} ( {u}_{out} , {i}_{in} , t + {τ}^{′} ( x , {i}_{in} , {𝓏}_{t} + Δ 𝓏 , {c}_{0} ) + τ ( x + Δ x , {u}_{out} , {𝓏}_{l} + Δ z , {c}_{0} ) )$$

in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, iᵢₙ, t) is the element of the experimental reflection matrix Rᵤᵢ(t) recorded by the transducer at spatial position Wout following the transmission of index iᵢₙ in the transmission basis (i), at time t,
c₀ being the expected speed of sound.

According to one variant, the de-scanned reflection matrix R′ is computed from a focused reflection matrix R by the following formula:

$${R}^{′} ( \{ Δ x , Δ x \} , \{ x , t \} ) = R ( x + Δ x , x , t , {𝓏}_{t} + Δ 𝓏 )$$

in which:
the de-scanned reflection matrix R′ is formed such that each row is composed of elements of a pair {Δx, Δz} and each column is composed of elements of a pair {x, t}, and
the focused reflection matrix R is calculated from the experimental reflection matrix Rᵤᵢ(t) by
the following formula:

$$R ( {x}_{out} , {x}_{in} , t , {𝓏}_{t} + Δ 𝓏 ) = \frac{1}{{N}_{in} {N}_{out}} {\sum }_{{i}_{in} , {u}_{out}} {R}_{ui} ( {u}_{out} , {i}_{in} , t + {τ}^{′} ( {x}_{in} , {i}_{in} , {𝓏}_{t} + Δ 𝓏 , {c}_{0} ) + τ ( {x}_{out} , {u}_{out} , {𝓏}_{t} + Δ 𝓏 , {c}_{0} ) )$$

in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, iᵢₙ, t) is the element of the experimental reflection matrix Rᵤᵢ(t) recorded by the transducer at spatial position uₒᵤₜ following the transmission of index iᵢₙ in the transmission basis (i) and at time t,
c₀ being the expected speed of sound.

According to one variant, a video is formed from a sequence of ultrasound images produced over a first number N of iterations of the steps of the method.

According to one variant:
among the N iterations, a second number P (smaller than N) of iterations do not include the steps of determining the de-scanned reflection matrix R′, the spatio-temporal averaging steps or the extraction steps, and
for these P iterations, in the ultrasound image formation step, the ultrasound image is obtained from an optimal position offset extracted during a previous iteration.

The present description relates, according to a second aspect, to a system for ultrasonic characterisation of a medium, configured for implementing methods such as those described above. The system according to the second aspect comprises:
an array of transducers adapted to generate a series of incident ultrasonic waves in a region of the medium, and to record over time the ultrasonic waves backscattered by said region; and
a computing unit associated with the transducer array and adapted to implement the method according to the first aspect.

The present description relates, according to a third aspect, to a method for ultrasonic characterisation of a medium, the method comprising:
a step of generating a series of incident ultrasonic waves USᵢₙ in a region of said medium by means of a transducer array, the series of incident ultrasonic waves forming a transmission basis i; and
a step of generating an experimental reflection matrix Rᵤᵢ(t) defined between the transmission basis i at the input and a reception basis u at the output, the coefficients of this matrix corresponding to the signals received by the transducers induced by the reflected ultrasonic waves.

The method is characterised in that it further comprises:
a step of determining a de-scanned reflection matrix R′,
which comprises, for a set of points of the medium of abscissa x and echo time t,
responses of the medium calculated by beamforming according to a sound speed model c₀ from the experimental reflection matrix Rᵤᵢ(t), between an input virtual transducer of spatial position rᵢₙ=(x, zₜ) and an output virtual transducer of spatial position rₒᵤₜ=(x+Δx, zₜ), both virtual transducers being at the same depth zₜ, zₜ being the depth of the isochronous volume expected for the sound speed c₀, the isochronous volume being the set of points of the medium contributing to the signals received at time t,
the responses of the de-scanned reflection matrix R′ being determined for a set of lateral offset values Δx including at least Δx=0 and for a set of sound speed models c₀, said de-scanned reflection matrix being formed such that each row corresponds to an element of a pair {Δx, c₀} which corresponds to a focal spot image, and each column corresponds to an element of a pair {x, t}, which is written as:

$${R}^{′} = [ {R}^{′} ( \{ Δ x , {c}_{0} \} , \{ x , t \} ) ]$$

an extraction step in which, from each focal spot image, an optimal lateral offset Δxₒₚₜ and an optimal sound speed model c̅opt, close to the maximum in said focal spot image, are determined.

Thanks to these arrangements, the method advantageously enables the medium to be probed locally at any point and in any direction with respect to the ballistic propagation time of the ultrasonic wave in the medium.

This method provides access to local information about the medium, making it possible to characterise the ultrasonic wave focusing process locally, and thus to estimate the sound speed distribution within the medium. This physical parameter is a particularly relevant biomarker in ultrasound imaging because it enables the diagnosis of certain diseases, such as, for example, hepatic steatosis.

This estimation of the sound speed is also very useful for improving the focusing and quality of the ultrasound image. This optimisation of ultrasound images is performed computationally, without requiring further transmissions and/or acquisitions, which is a significant advantage, especially for in vivo measurements.

In various embodiments of the method, one and/or the other of the following arrangements may also possibly be used.

According to one variant, the method further comprises:
a step of forming an ultrasound image by computing the reflectivity at a plurality of points in the medium, from the de-scanned reflection matrix R′, the reflectivity of each of these points in the medium corresponding to the response of the medium between the input virtual transducer at spatial position rᵢₙ=(x, zₜ) and the output virtual transducer at spatial position rₒᵤₜ=(x+Δxₒₚₜ, zₜ), zₜ being the depth of the isochronous volume expected for the optimal sound speed model c̅opt and said reflectivity being defined by:

$${ℐ}_{2} ( x , t ) = \vert {R}^{′} ( \{ Δ x = Δ {x}_{opt} ( x , t ) , {c}_{0} = {\overline{c}}_{opt} ( x , t ) , \{ x , t \} ) \vert .$$

According to one variant, the ultrasound image is transformed in the depth dimension, using the optimal sound speed model c̅opt by:

$${\tilde{ℐ}}_{2} ( x , \tilde{𝓏} ) = {ℐ}_{2} ( x , t = 2 \tilde{𝓏} / {\overline{c}}_{opt} ( x , t ) ) ,$$

in which \tilde{z} is the estimated depth z of a pixel of the ultrasound image.

According to one variant, the ultrasound image is displayed on a display device.

According to one variant:
local sound speeds c(r), are calculated, for a set of pairs {x, t}, from the optimal sound speed values c̅opt obtained for the focal spot images of said pairs {x, t}.

According to one variant, the method further comprises a step of forming a local sound speed image of the medium from said set of local sound speed values, and said local sound speed image is displayed on a display device.

According to one variant, the de-scanned reflection matrix R′ is recalculated using the local sound speed values.

According to one variant, the method further comprises, prior to the extraction step:
a step of spatio-temporal averaging of the de-scanned reflection matrix R′ in which the values of the de-scanned reflection matrix are averaged locally along the lateral dimension x and the echo time t, for each element of a pair {Δx, c₀}, the focal spot images then being averaged.

According to one variant:
in the spatio-temporal averaging step, an incoherent point spread function PSF_{inc} is calculated by the following formula:

$${PSF}_{inc} ( \{ Δ x , {c}_{0} \} , \{ x , t \} ) = \sqrt{{( {\vert {R}^{′} ( \{ Δ x , {c}_{0} \} , \{ {x}^{′} , {t}^{′} \} ) W ( {x}^{′} - x , {t}^{′} - t ) \vert}^{2} )}_{[ {x}^{′} , {t}^{′} ]}}$$

for a set of lateral offset values Δx including at least Δx=0 and for a set of sound speed model values c₀, in order to obtain the focal spot image for the lateral position x and the echo time t,
in which:
<.> is an averaging operator with respect to the parameters of the pair {x′, t′}
R′ is the de-scanned reflection matrix,
W(x′, t′) is a spatio-temporal weighting window using the same parameters, and
in the extraction step, a maximum point of the focal spot image is sought, corresponding to a maximum of the magnitude of the points of the focal spot image, and the optimal sound speed model c̅opt is the ordinate of said maximum point in said focal spot image.

According to one variant:
in the spatio-temporal averaging step, a singular value decomposition (SVD) of a local focal spot matrix R^{(l)} is computed, said local focal spot matrix being derived from the de-scanned reflection matrix R′ by:

$${R}^{( l )} ( \{ x , t \} ) = [ {R}^{′} ( \{ Δ x , {c}_{0} \} , \{ {x}^{′} , {t}^{′} \} ) W ( {x}^{′} - x , {t}^{′} - t ) ]$$

in which:
R′ is the de-scanned reflection matrix,
W(x′, t′) is a spatio-temporal weighting window using the same parameters.
   The singular value decomposition (SVD) of said local matrix is written as:

$${R}^{( l )} ( x , t ) = {\sum }_{p} {λ}_{p} {U}_{p} ( \{ x , t \} ) {V}_{p}^{†} ( \{ x , t \} )$$

in which:
λₚ are the singular values of said local matrix,
Uₚ are the output eigenvectors,
Vₚ are the input eigenvectors,
a coherent point spread function PSF_{coh} is computed by the following formula:

$${PSF}_{coh} ( \{ Δ x , {c}_{0} \} , \{ x , t \} ) = {U}_{1} ( \{ Δ x , {c}_{0} \} , \{ x , t \} )$$

for a set of lateral offset values Δx including at least Δx=0 and for a set of sound speed model values c₀, in order to obtain the focal spot image for the lateral position x and the echo time t, and
in the extraction step, a maximum point of the focal spot image is sought, corresponding to:
a maximum of the magnitude of the points of the focal spot image, or
a maximum of the magnitude of the derivative with respect to the sound speed c₀ of the points of the focal spot image, or
a maximum of the magnitude of the derivative with respect to the sound speed c₀ of the imaginary part of the points of the focal spot image, and
   the optimal sound speed model c̅opt is the ordinate of said maximum point in said focal spot image.

According to one variant, in the extraction step, the lateral offset Δxₒₚₜ is considered to be zero, Δxₒₚₜ=0.

According to one variant, in the step of determining a de-scanned reflection matrix R′, the de-scanned reflection matrix R′ is formed only for lateral offset values Δx equal to zero, Δx=0.

According to one variant, in the spatio-temporal averaging step, the values of the de-scanned reflection matrix R′ are averaged to determine a focal spot image, for each element of a pair {Δx=0, Δz}, along the lateral dimension x and the echo time t.

According to one variant, in the extraction step, the optimal sound speed model c̅opt is determined from the points of the focal spot image having a lateral offset Δx equal to zero, Δx=0.

According to one variant, the beamforming calculations between the input virtual transducer and the output virtual transducer are performed based on the experimental reflection matrix Rᵤᵢ(t), using a forward delay of the ultrasonic waves between the transmission basis (i) and the input virtual transducer, and using a return delay of the ultrasonic waves between the output virtual transducer and the transducers of the reception basis (u).

According to one variant, the de-scanned reflection matrix R′ is calculated from the experimental reflection matrix Rᵤᵢ by the following formula:

$${R}^{′} ( \{ Δ x , {c}_{0} \} , \{ x , t \} ) = \frac{1}{{N}_{in} {N}_{out}} {\sum }_{{i}_{in} , {u}_{out}} {R}_{ui} ( {u}_{out} , {i}_{in} , t + {τ}^{′} ( x , {i}_{in} , {z}_{t} , {c}_{0} ) + τ ( x + Δ x , {u}_{out} , {z}_{t} , {c}_{0} ) )$$

in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, iᵢₙ, t) is the element of the experimental reflection matrix Rᵤᵢ(t) recorded by the transducer at spatial position Wout following the transmission of index iᵢₙ in the transmission basis (i), at time t,
c₀ being the expected speed of sound.

According to one variant, the de-scanned reflection matrix R′ is computed from a focused reflection matrix R by the following formula:

$${R}^{′} ( \{ Δ x , {c}_{0} \} , \{ x , t \} ) = R ( x + Δ x , x , t , {c}_{0} )$$

in which:
the de-scanned reflection matrix R′ is formed such that each row is composed of elements of a pair {Δx, Δz} and each column is composed of elements of a pair {x, t}, and
the focused reflection matrix R is calculated from the experimental reflection matrix Rᵤᵢ(t) by
the following formula:

$$R ( {x}_{out} , {x}_{in} , t , {c}_{0} ) = \frac{1}{{N}_{in} {N}_{out}} {\sum }_{{i}_{in} , {u}_{out}} {R}_{ui} ( {u}_{out} , {i}_{in} , t + {τ}^{′} ( {x}_{in} , {i}_{in} , {𝓏}_{t} , {c}_{0} ) + τ ( {x}_{out} , {u}_{out} , {𝓏}_{t} , {c}_{0} ) )$$

in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, iᵢₙ, t) is the element of the experimental reflection matrix Rᵤᵢ(t) recorded by the transducer at spatial position uₒᵤₜ following the transmission of index iᵢₙ in the transmission basis (i) and at time t,
c₀ being the expected speed of sound.

According to one variant, a video is formed from a sequence of ultrasound images produced over a first number N of iterations of the steps of the method.

According to one variant, among the N iterations, a second number P (strictly less than N) of iterations do not include the steps of determining the de-scanned reflection matrix R′, the spatio-temporal averaging steps or the steps of extracting the optimal sound speed model, and for these P iterations, in the ultrasound image formation step, the ultrasound image is obtained from an optimal sound speed extracted during a previous iteration.

The present system relates, according to a fourth aspect, to a system for ultrasonic characterisation of a medium, configured for implementing methods such as those described above. The system according to the fourth aspect comprises:
an array of transducers adapted to generate a series of incident ultrasonic waves in a region of the medium, and to record over time the ultrasonic waves backscattered by said region; and
a computing unit associated with the transducer array and adapted to implement the method according to the first aspect.

### BRIEF DESCRIPTION OF FIGURES

Other advantages and features of the technique presented above will be seen in the detailed description below, presented in a non-limiting manner for illustration purposes, with reference to the figures in which:

FIG. 1, (a) to (d), (already described) illustrates the impact of aberrations in ultrasound imaging, according to the prior art;

FIG. 2, (a) to (f), (already described) illustrates known transmission/reception sequences for ultrasound imaging and quantification;

FIG. 3 illustrates conventional confocal imaging and matrix imaging;

FIG. 4 illustrates an example of an ultrasound characterisation system for implementing the methods according to the present description;

FIG. 5 illustrates the definitions used in the method for forming an ultrasound image according to the present description;

FIG. 6, (A) to (D), illustrates a focusing error due to an incorrect sound speed value;

FIG. 7, (a) to (d), illustrates the impact of an incorrect sound speed value on an ultrasound image obtained from simulated ultrasound data;

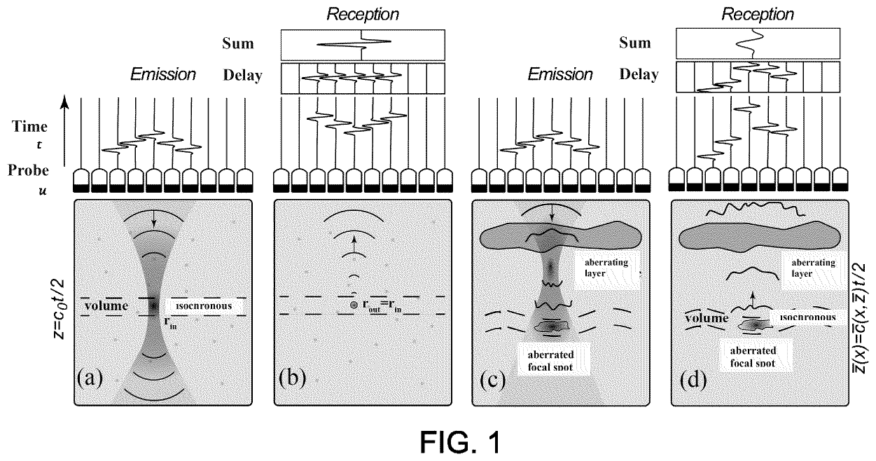

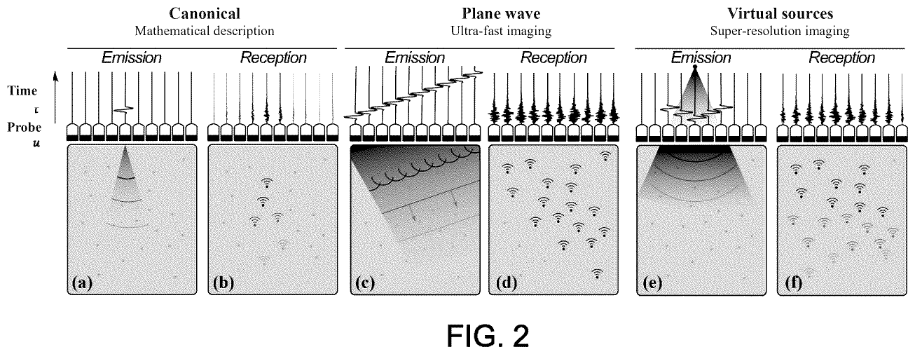

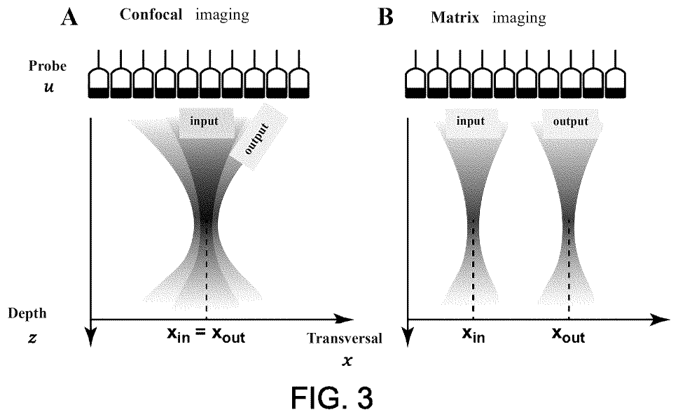

FIG. 8:
(a1) to (a3) show three experimental configurations with different media: a first without an aberrating layer, a second with an aberrating water layer, and a third with an aberrating plexiglass layer;
(b1) to (b3) show the ultrasound images obtained without the method of the present disclosure, corresponding respectively to the media in (a1) to (a3);
(c) shows the histogram of the estimated focusing errors for the different experimental configurations in (a1) to (a3);
(d1) to (d3) show the ultrasound images obtained with a first embodiment of the method, which corrects a focusing error, corresponding respectively to the media in (a1) to (a3);

FIG. 9, (a) to (d), illustrates the method for correcting the focusing error;

FIG. 10:
(a) to (c) shows focal spot images (magnitudes) obtained without spatio-temporal averaging in (a), and then with different spatio-temporal averaging variants for the first embodiment of the method for correcting the focusing;
(d) shows the imaginary part of the spatio-temporal averaging variant in (c);
(e) shows a curve of the axial evolution of the phase of the focal spot image in (c);
(f) shows a curve of the axial evolution of the imaginary part of the focal spot image in (c);
(g) shows curves for determining the axial focusing error on the focal spot images in (b) to (d);

FIG. 11:
(a) shows an ultrasound image of a liver without implementation of the method of the present disclosure;
(b) shows a map of the focusing error (optimal depth/axial offset) determined by the method of the first embodiment corresponding to the image in (a);
(c) shows a map of the optimal integrated sound speed determined by the method of the second embodiment corresponding to the image in (a);
(d) shows a local sound speed map obtained from the map in (c);

FIG. 12:
(a) shows an ultrasound image of a liver without implementation of the method of the present disclosure;
(b) shows a corrected ultrasound image of the image in (a) obtained with the method of the first embodiment;
(c) and (d) show enlarged regions of the image in (a);
(e) and (f) show the same enlarged regions in the corrected image in (b);

FIG. 13:
(a) shows an ultrasound image of a liver without implementation of the method of the present disclosure;
(b) shows the focused reflection matrix associated with the isochronous volume represented by the grey arc on the ultrasound image in (a);
(c) shows a corrected ultrasound image of the liver in the image in (a) obtained by the method of the first embodiment of the focus correction process;
(d) shows the focused reflection matrix associated with the isochronous volume represented by the grey arc on the corrected ultrasound image in (c);
(e) shows the transverse evolution of the focal spot derived from the focused reflection matrices shown in (b) and (d);

FIG. 14:
(a) to (b) show focal spot images (magnitudes) obtained with different spatio-temporal averaging variants for the second embodiment of the sound speed correction process;
(c) shows the imaginary part of the focal spot image shown in (b);
(d) shows curves for determining the axial maximum on the focal spot images shown in (a) to (c);

FIG. 15:
(a) shows an ultrasound image with the focusing errors (axial/depth offsets obtained) overlaid;
(b) shows a corrected ultrasound image of the liver produced by the method of the second embodiment of the sound speed correction process;
(c) shows a corrected and repositioned ultrasound image derived from the image in (a) by the method of the second embodiment of the sound speed correction process;

FIG. 16:
(a) shows an ultrasound image of a liver without implementation of the method of the present disclosure;
(b) shows the focused reflection matrix associated with the isochronous volume represented by the grey arc on the ultrasound image in (a);
(c) shows a corrected ultrasound image of the liver obtained by the method of the second embodiment of the sound speed correction process;
(d) shows the focused reflection matrix associated with the isochronous volume represented by the grey arc on the corrected ultrasound image in (c);

In the various embodiments described with reference to the figures, similar or identical elements have the same references unless otherwise stated.

### DETAILED DESCRIPTION

In the detailed description that follows, only certain embodiments are described in detail in order to ensure clarity of explanation, but these examples are not intended to limit the general scope of the principles set out herein.

The various embodiments and aspects described in the present description may be combined or simplified in multiple ways. In particular, the steps of the different methods may be repeated, interchanged and/or performed in parallel, unless otherwise specified.

The present description relates to methods and systems for ultrasonic characterisation of a medium, and applies in particular to medical imaging of living or non-living tissues. The medium may, for example, be a heterogeneous medium that needs to be characterised, for instance to identify and/or characterise its heterogeneities. In some cases, these methods and systems may be applied to non-destructive testing of products, such as mineral, metallic, or other components. These characterisation techniques are known to be non-invasive for the medium, which is therefore preserved.

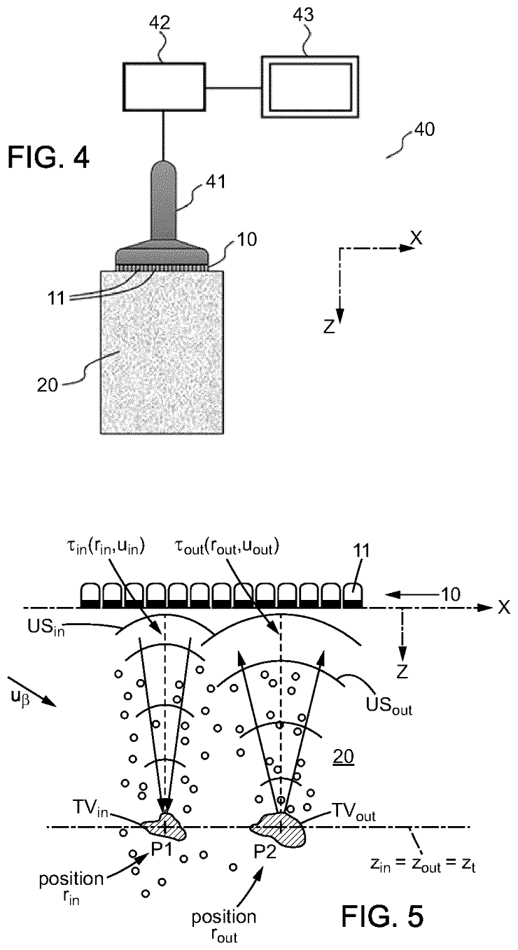

FIG. 4 illustrates an example of an ultrasound imaging system 40 for implementing methods for ultrasound imaging of a medium such as a heterogeneous medium 20, according to the present description. This system and method are useful for enabling the formation of an ultrasound image of at least a portion (region of interest or field of view) of the medium. The system 40 comprises at least one array 10 of transducers 11, for example a linear, two-dimensional, or matrix array; the transducers are, for example, ultrasonic piezoelectric transducers, which may typically take the form of a rigid bar placed in direct or indirect contact with the medium 20. The transducer array may, for example, form part of a probing device 41 (commonly called a probe); the transducer array is connected to a computing unit 42, which may itself be linked to or associated with a display device 43; the computing unit transmits and records electrical signals to and/or from each of the transducers 11. The ultrasonic transducers then convert these electrical signals into ultrasonic waves and vice versa. A “connection” or “link” between the probing device 41, the computing unit 42, and the display device 43 refers to any type of wired connection, whether electrical or optical, or any type of wireless connection using any protocol such as WiFi™, Bluetooth™ or others. These connections or links may be unidirectional or bidirectional. The associated display device 43 may be of any type, such as a touch or non-touch screen, connected or not.

The computing unit 42 is configured for the implementation of computation or processing steps, in particular for the implementation of steps of methods according to the present description. By convention, a spatial coordinate system is defined for the medium 20, using a first axis X and a second axis Z perpendicular to the first axis. For simplicity, the first axis X corresponds to the transverse direction in which the transducers 11 are aligned for a linear array, and the second axis Z corresponds to the depth of the medium 20 relative to this array 10 of transducers 11. This definition may be adapted to the context and thus, for example, extended to a three-axis spatial coordinate system in the case of a two-dimensional array 10, or to a polar coordinate system in the case of a curved array 10, or to any other suitable coordinate system adapted to and/or dependent on the shape of the ultrasonic transducer array 10. Accordingly, in the remainder of the present description, a Cartesian XZ coordinate system, corresponding to a linear probe, will be used to simplify this description, but a person skilled in the art would generalise or apply the results to any type of coordinate system.

In FIG. 4 as in the remainder of the description, reference is made to a transducer array for transmission and reception, it being understood that, in a more general case, several transducer arrays may be used simultaneously. Similarly, an array may consist of one (1) to N transducers, either identical in type or of different kinds. The transducers may operate both as a transmitter and then as a receiver, or in some cases only as a transmitter and in other cases only as a receiver.

The transducer array may, for example, serve both as a transmitter and as a receiver, or consist of several transducer sub-arrays, some dedicated to transmitting and others to receiving the ultrasonic waves. “Transducer array” refers to at least one transducer, an aligned or non-aligned series of transducers, or a matrix of transducers.

When reference is made in the present description to computation or processing steps for implementing method steps in particular, it is understood that each computation or processing step may be implemented by software, hardware, firmware, microcode, or any appropriate combination of these technologies or closely related technologies. When software is used, each computation or processing step may be implemented by computer program instructions or code that may, for example, be interpreted or executed. These instructions may be stored or transmitted to a storage medium readable by a computer (or a computing unit) and/or be executed by a computer (or computing unit) in order to implement these computation or processing steps.

### Analysis of a Point of the Medium Via a Focused Reflection Matrix

The present description describes methods and systems for ultrasonic characterisation of a medium. In practical cases, the medium is assumed to be heterogeneous. These methods and systems are based on the definitions shown in FIG. 5:

The following are defined in the medium:
a first point P1 of expected spatial position rᵢₙ in the spatial coordinate system of the medium, and
a second point P2 of expected spatial position rₒᵤₜ in the spatial coordinate system of the medium.

The spatial positions rᵢₙ and rₒᵤₜ are written in bold to indicate that these elements are position vectors, vectors taken in the spatial coordinate system of the medium (X, Z). Other representations and definitions of the positions of the points are possible and accessible to any ultrasound technician.

In the present disclosure, the first point P1 has a lateral position denoted xᵢₙ. The second point P2 has a lateral position denoted xₒᵤₜ. The two points P1 and P2 have the same expected depth zᵢₙ=zₒᵤₜ, also denoted z, which is controlled by the echo time t considered. For a medium with a homogeneous sound speed, this depth z would be equal to the expected depth zt=c₀t/2. Thus, the spatial positions of points P1 and P2 are respectively rᵢₙ=(xᵢₙ, z) and rₒᵤₜ=(xₒᵤₜ, Z).

These two points P1 and P2 are preferably chosen at a short distance from each other, i.e. a few millimetres apart, for example twenty (20) millimetres or less.

As shown in FIG. 5, the ultrasonic characterisation method implemented by the computing unit 42 of the system 40 comprises:
a step of generating a series of incident ultrasonic waves USᵢₙ in a region of said medium, by means of an array 10 of transducers 11, said series of incident ultrasonic waves forming a transmission basis i; and
a step of generating (constructing/measuring) an experimental reflection matrix Rᵤᵢ(t) defined between the transmission basis i at the input and a reception basis u at the output;
a step of determining a focused reflection matrix Rₓₓ(t, z, c₀) which comprises responses of the medium between an input virtual transducer TVᵢₙ of spatial position rᵢₙ and an output virtual transducer TVₒᵤₜ of spatial position rₒᵤₜ.

This focused reflection matrix Rₓₓ can be expressed in different ways. In the expressions in the present disclosure, the first point P1 of spatial position (xᵢₙ, z) is taken as the reference. However, these expressions may be established relative to the second point P2 of spatial position (xₒᵤₜ, Z) or relative to a midpoint between P1 and P2, of spatial position ((xᵢₙ+xₒᵤₜ)/2, z) or relative to any reference point. A person skilled in the art will be able to make the necessary variable changes in the expressions presented here.

The responses of the medium are computed by beamforming from the experimental reflection matrix Rᵤᵢ(t).

The responses of the focused reflection matrix Rₓₓ(t, z, c₀) correspond to an acoustic pressure field computed between all points in the medium of lateral positions xᵢₙ, and xₒᵤₜ, located at the expected depth z and at an echo time t, and for an assumed sound speed c₀. In other words, this focused reflection matrix Rₓₓ(t, z, c₀) is defined by: Rₓₓ(t, z, c₀)=[R(xₒᵤₜ, xᵢₙ, t, z, c₀)]

The parameters constituted by the depth z in the medium and the sound speed c₀ control the delay laws used in the beamforming process (i.e. the focusing process).

The transmission basis i at the input is, for example, a basis consisting of waves each generated by only one of the transducers 11 in the array 10, or a basis consisting of plane waves with an angular inclination θ relative to the x-axis, or a basis consisting of virtual sources, as described previously in the description of FIGS. 2(a) to 2(f).

The reception basis u is, for example, the basis of the transducers 11. Optionally, another reception basis may be used, for example a frequency-domain or spectral basis.

Thus, the ultrasonic wave generation step is to be understood to be between the transmission basis i and the reception basis u. This ultrasonic generation step is therefore defined for any type of ultrasonic waves, focused or unfocused, such as plane waves.

In the matrix generation step, the experimental reflection matrix Rᵤᵢ(t) est defined between the transmission basis i at the input and a reception basis u at the output. This matrix contains all the time-domain responses of the medium, measured at time t by each transducer 11 of spatial coordinate Wout and for each transmission lin. It will be understood that the elements identified by the index “in” refer to transmission (i.e. the input) and those identified by the index “out” refer to reception (i.e. the output). This experimental matrix may also be recorded and/or stored, for example in the memory of the computing unit, or on any other support, removable or not, allowing permanent or temporary storage.

More precisely, in the step of determining the focused reflection matrix Rₓₓ(t, z, c₀), the following are applied:
an input focusing process based on the experimental reflection matrix Rᵤᵢ(t) which uses the forward time-of-flight of the waves between the transmission basis i and the input virtual transducer TVᵢₙ and which creates an “input” focal spot around the first point P1 of spatial position rᵢₙ, said input focal spot corresponding to the input virtual transducer TVᵢₙ,
an output focusing process based on the experimental reflection matrix Rᵤᵢ(t) which uses the return time-of-flight of the waves between the output virtual transducer TVₒᵤₜ and the transducers of the reception basis u and which creates an “output” focal spot around the second point P2 of spatial position rₒᵤₜ, said output focal spot corresponding to the output virtual transducer TVₒᵤₜ.

These input and output focusing processes in fact form an input-output focusing process, referred to simply as the focusing process in the remainder of this description.

In other words, in this ultrasonic characterisation method, the input virtual transducer TVᵢₙ corresponds to an ultrasonic “virtual source” at the spatial position rᵢₙ in the medium, and the output virtual transducer TVₒᵤₜ corresponds to an ultrasonic “virtual sensor” located at the spatial position Tout. This virtual source and this virtual sensor are spatially separated by the difference in their spatial positions Δr=rₒᵤₜ−rᵢₙ. In the present case, they are simply separated laterally by Δx=xₒᵤₜ−xᵢₙ. Their expected depth is the parameter z used in the focusing law for a sound speed model c₀. Their actual depth is dictated by the axial (depth) position of the isochronous volume, i.e. by the echo time t and by the distribution of the sound speed c(r) within the medium. The lateral dimension of the virtual transducers is dictated by the focal spot produced by focusing at this actual depth.

For example, computing the focused reflection matrix Rₓₓ(t, z, c₀) of the medium between the input virtual transducer TVᵢₙ and the output virtual transducer TVₒᵤₜ via said input and output focusing processes is an improved beamforming method which can be expressed by the following simplified formula:

$$R ( {x}_{out} , {x}_{in} , t , 𝓏 , {c}_{0} ) = \frac{1}{{N}_{in} {N}_{out}} {\sum }_{{i}_{in} , {u}_{out}} {R}_{ui} ( {u}_{out} , {i}_{in} , t + {τ}^{′} ( {x}_{in} , {i}_{in} , {𝓏}_{t} , {c}_{0} ) + τ ( {x}_{out} , {u}_{out} , 𝓏 , {c}_{0} ) ) \qquad ( 1 )$$

in which:
Nᵢₙ is the number of elements in the transmission basis i,
Nₒᵤₜ is the number of elements in the reception basis u at the output,
R(uₒᵤₜ, iᵢₙ, t+τ′(xᵢₙ, iᵢₙ, c₀)+τ(xₒᵤₜ, uₒᵤₜ, z, c₀) is the element of the experimental reflection matrix Rᵤᵢ(t) recorded by the transducer Wout following the transmission iᵢₙ to which the delay times τ and τ′ have been applied, and
τ′(xᵢₙ, iᵢₙ, c₀) is the delay or time shift that must be applied on transmission to cause each incident wave iᵢₙ to interfere constructively at the first focal point having the expected spatial position (xᵢₙ, z) for a sound speed model c₀. This time shift is derived from the forward time-of-flight of the ultrasonic wave between the transducers of the transmission basis i and the input virtual transducer TVᵢₙ having the expected spatial position rᵢₙ (first point P1).

τ(xₒᵤₜ, uₒᵤₜ, z, c₀) is the delay or time shift that must be applied to the signals measured by each output transducer uₒᵤₜ to cause constructive interference of the echoes originating from a scatterer located at the expected spatial position (xₒᵤₜ, z) for the same sound speed model c₀. This time shift is derived from the return time-of-flight of the ultrasonic wave between the output virtual transducer TVₒᵤₜ having the spatial position rₒᵤₜ (second point P2) and the transducers of the reception basis u.

These delays τ and τ′ are computed from a sound speed model. The simplest assumption is a homogeneous medium with a constant sound speed c₀. In that case, the time-of-flight values are directly obtained from the distances between the transducers of the probe and the virtual transducers.

For example, in the specific case of a plane wave with a transmission angle θᵢₙ:
the transmission delay τ′ can be obtained by:

$${\underbrace{τ ( {θ}_{in} , {r}_{in} , {c}_{0} )}}_{Retard} = {\underbrace{{t}_{to ⌈} ( {θ}_{in} , {r}_{in} , {c}_{0} )}}_{Temps de vol} - \frac{{𝓏}_{in}}{{c}_{0}} = \frac{{x}_{in} sin ( {θ}_{in} ) + {𝓏}_{in} cos ( {θ}_{in} )}{{c}_{0}} - \frac{{𝓏}_{in}}{{c}_{0}}$$

in which zᵢₙ/c₀ is the time taken to reach the input virtual transducer.
the reception delay τ can be obtained by:

$${\underbrace{τ ( {u}_{out} , {r}_{out} , {c}_{0} )}}_{Retard} = {\underbrace{{t}_{tof} ( {u}_{out} , {r}_{out} , {c}_{0} )}}_{Temps de vol} - \frac{{𝓏}_{out}}{{c}_{0}} = \frac{\sqrt{{( {x}_{out} - {u}_{x} )}^{2} + {𝓏}_{out}^{2}}}{{c}_{0}} - \frac{{𝓏}_{out}}{{c}_{0}}$$

in which zₒᵤₜ/c₀ is the time taken to reach the output virtual transducer.

These examples of delay calculations thus clearly show that they depend on the type of wave and on the sound speed, assumed here to be constant within the medium.

The number of elements in the transmission basis Nᵢₙ is, for example, greater than or equal to one (1), and advantageously greater than or equal to two (2). The number of elements in the reception basis Nₒᵤₜ is, for example, greater than or equal to two (2).

This improved beamforming formula is therefore a double sum of the time-domain responses recorded in the experimental reflection matrix Rᵤᵢ: a first sum according to the transmission basis i representing transmission focusing, and a second sum according to the reception basis u associated with reception focusing. This computation is carried out for the spatial coordinates of the two points P1 and P2, i.e. the respective points with the expected spatial positions rᵢₙ=(xᵢₙ, z) and rₒᵤₜ=(xₒᵤₜ, Z). The result of this improved beamforming formula is therefore a time-domain signal for these two spatial coordinates (rᵢₙ, rₒᵤₜ) or for these two lateral positions xᵢₙ, xₒᵤₜ.

Such a beamforming formula can also be supplemented by input and output weighting terms, often referred to as transmission and/or reception apodisation.

The experimental reflection matrix Rᵤᵢ(t) recorded may be a “real” matrix, i.e. one consisting of real coefficients in the time domain, since the electrical signals recorded by each transducer are real numbers. Alternatively, this matrix may be a “complex” matrix, i.e. one consisting of complex values, for example in the case of demodulation for in-phase/quadrature beamforming (known as “IQ beamforming”).

It is thus possible to derive a line of the ultrasound image of the medium from this time-domain response after beamforming by considering the absolute value of the confocal signals characterised by identical lateral positions at the input and output (xᵢₙ=xₒᵤₜ) and at the ballistic depth z=zₜ=c₀t/2. Thus, to construct an ultrasound image, it is possible to scan or select a set of lateral positions x=xᵢₙ=xₒᵤₜ corresponding to a set of lines of the ultrasound image. The ultrasound image Ĩ can then be constructed from the diagonal coefficients of the focused reflection matrix, by:

$$\begin{matrix}\tilde{ℐ} ( x , {z}_{t} ) = ℐ ( x , t ) , or \\ ℐ ( x , t ) = \vert R ( x , x , t , {𝓏}_{t} = {c}_{0} t / 2 ) \vert\end{matrix} \qquad ( 2 )$$

The axial dimension of the ultrasound image is thus dictated by the time-of-flight t of the echoes. If the sound speed model c₀ matches the actual speed of sound in the medium, the ultrasound image provides a reliable estimate of the medium's reflectivity, allowing the depth of the scatterers to be assessed.

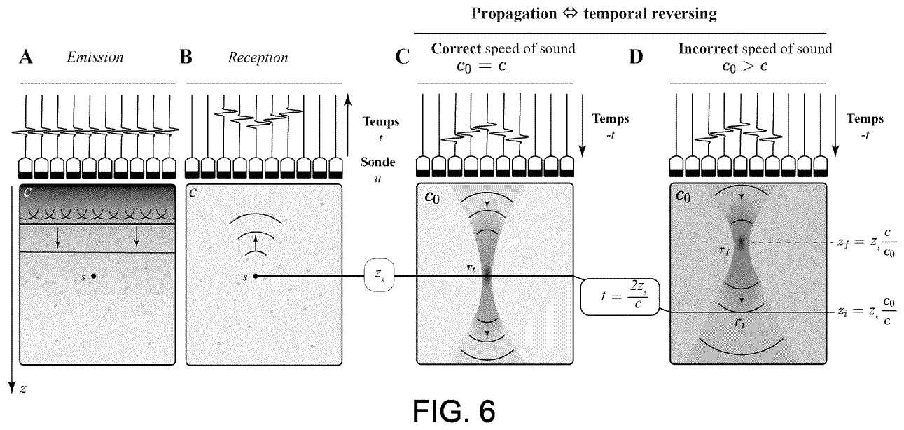

However, under real conditions (in vivo in medical imaging), the sound speed model c₀ often does not match the distribution of the sound speed c(r) in the medium, which can frequently be heterogeneous or even highly heterogeneous. To understand the impact that the mismatch between c₀ and c can have, the analogy between beamforming and a numerical time-reversal experiment can be illuminating. This experiment is illustrated in FIG. 6 where the goal is to use time reversal to back-propagate the wave scattered by a scatterer s in a medium with sound speed c, as shown in FIGS. 6(a) and 6(b), into a new medium with sound speed c₀.

If c=c₀, the refocusing plane, z=z_{f}, of the time-reversed wave coincides with the depth of the scatterer zₛ and with the position of the isochronous volume, zₜ=c₀t/2, as shown in FIG. 6(c).

For c<c₀ (or conversely c>c₀), the wave focusing takes place upstream (or conversely downstream) at a depth z_{f}, such that:

$${𝓏}_{f} = {𝓏}_{s} c / {c}_{0} \qquad ( 3 )$$

under the paraxial approximation, while the isochronous volume associated with the target's time-of-flight appears downstream (or conversely upstream) of the focal plane, at a depth zₜ, such that:

$${𝓏}_{t} = {c}_{0} {t}_{s} / 2 = {𝓏}_{s} - {𝓏}_{l} + {𝓏}_{l} {c}_{0} / {c}_{l} \qquad ( 4 )$$

Thus, the ultrasound image shows the scatterer at the incorrect depth (zₜ≠zₛ) and the image is degraded because the wave is completely defocused at the isochronous volume (z_{f}≠zₜ) as can be seen in FIG. 6(d). In other words, the depth of the isochronous volume zₜ, which depends on the time-of-flight t and the sound speed c0, is different from the focal depth z_{f} of the ultrasonic wave in this particular example.

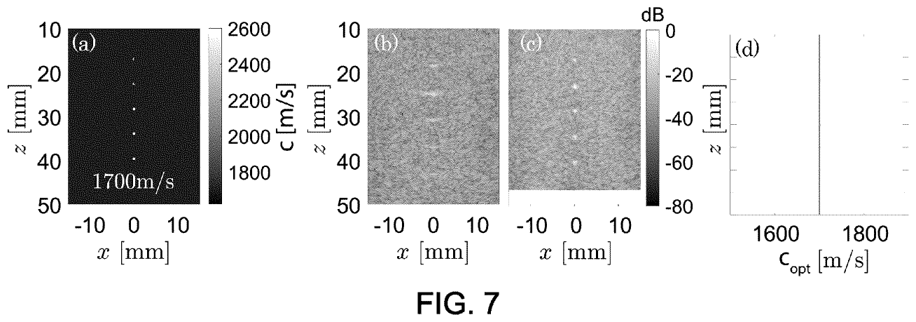

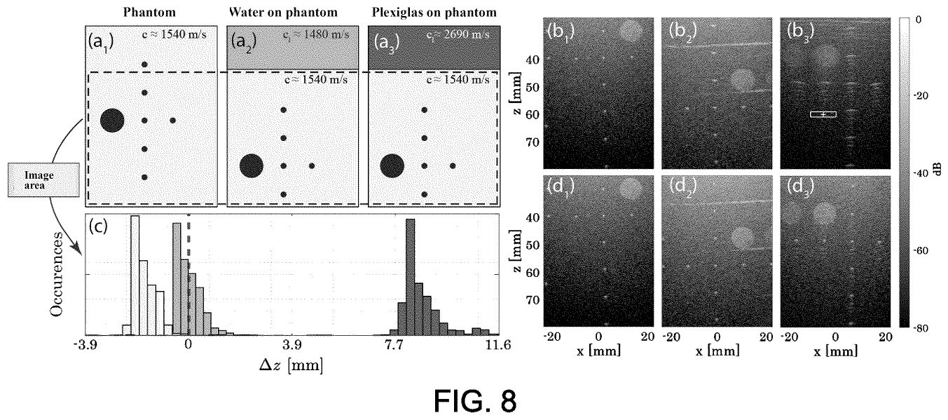

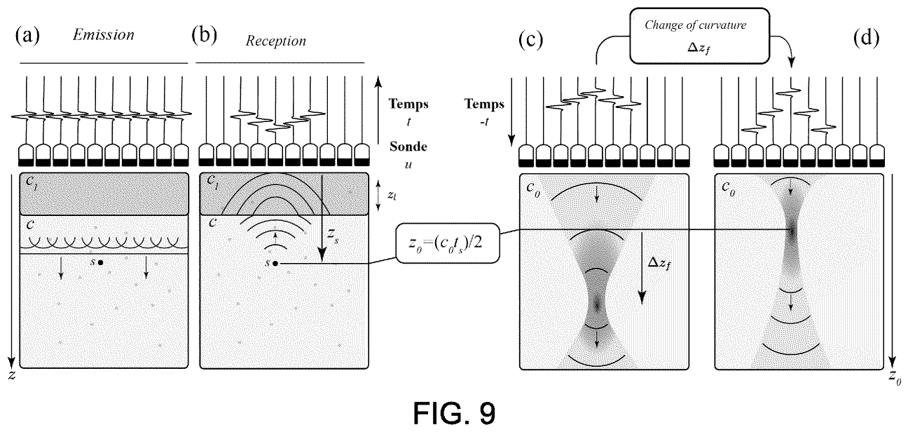

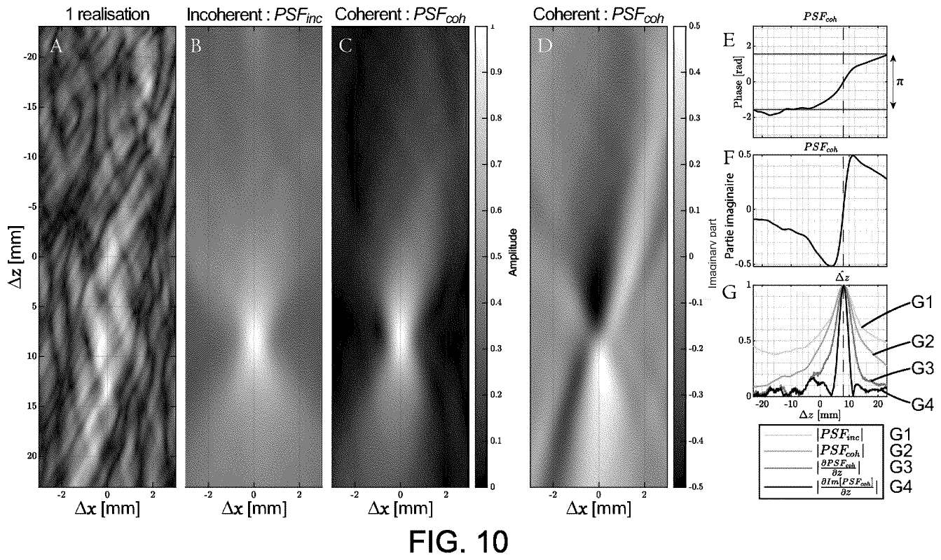

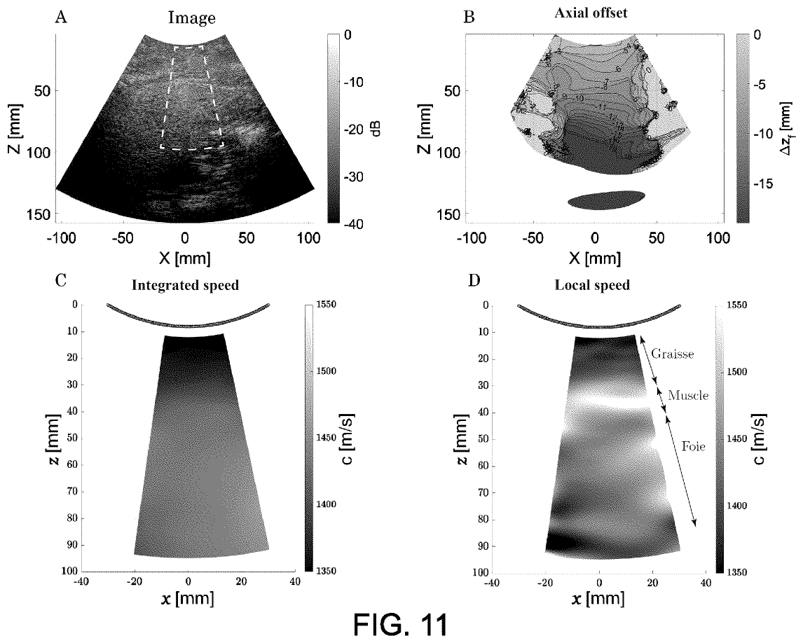

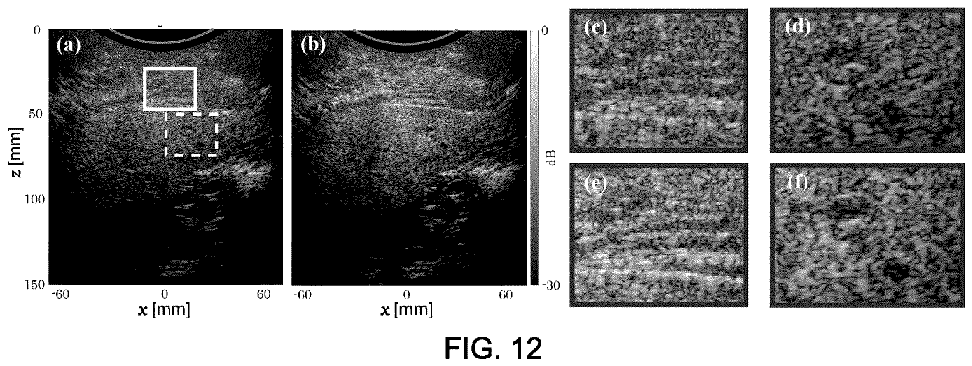

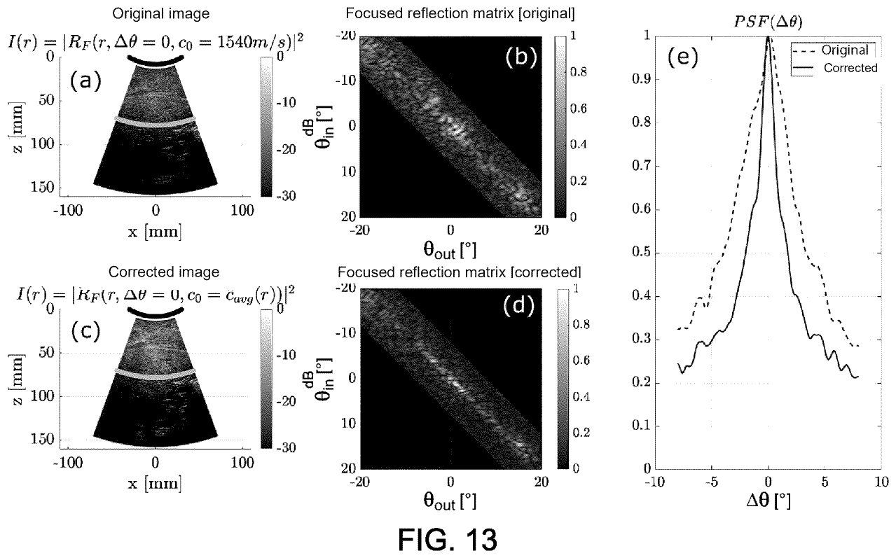

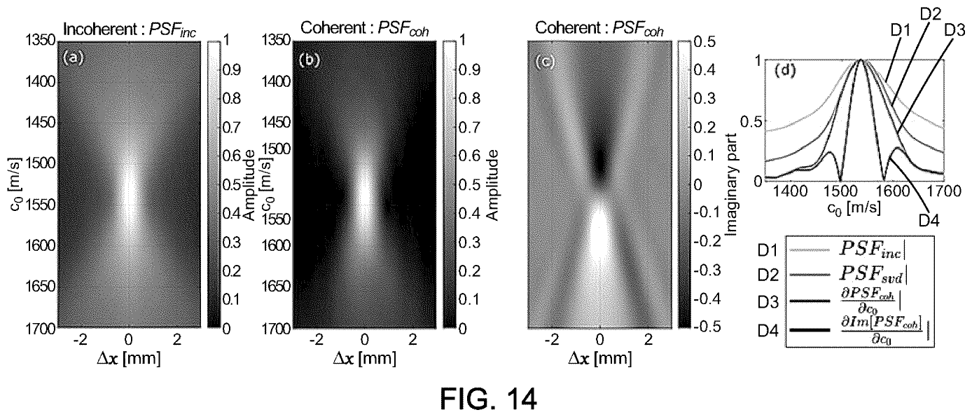

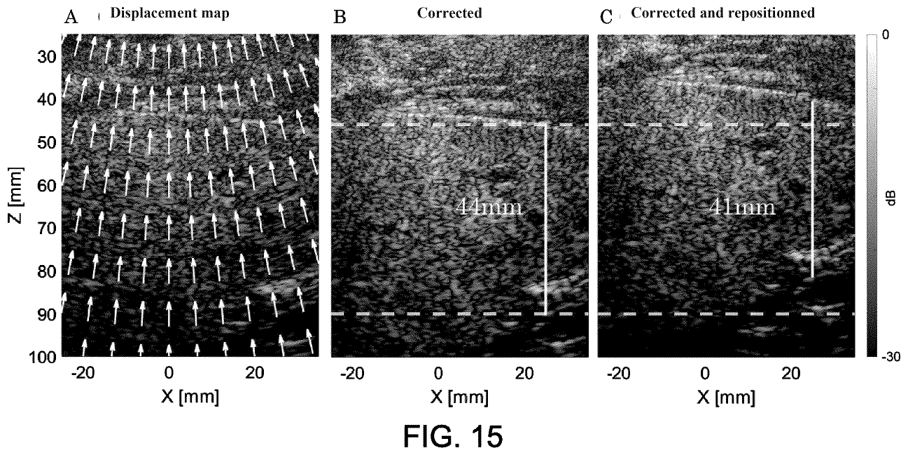

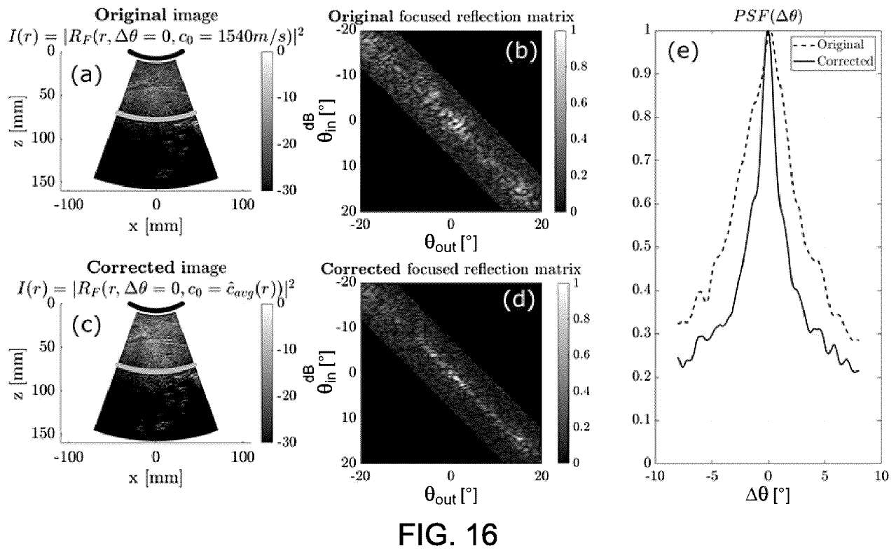

FIG. 7 illustrates the impact of an incorrect sound speed model on the ultrasound image by means of a k-Wave simulation of an acoustic phantom (c=1700 m/s) mimicking the properties of soft tissues (ultrasound speckle) and containing echogenic scatterers to allow the contrast and resolution of the image formed to be assessed.

If the sound speed model c₀ is incorrect, as shown in FIG. 7(b) in which a sound speed c₀ of 1850 m/s is used, the scatterers appear at the wrong depth compared to FIG. 7(a) which shows a model of the simulated medium. Furthermore, the mismatch between the focusing depth z_{f} and the depth of the isochronous volume z produces an enlarged and distorted focal spot, resulting in poor transverse/lateral resolution of each bright point in this image 7(b).

Thus, uncertainty exists for each ultrasound image regarding the axial dimension of the image, which is governed by the echo time t and which makes any distance measurement unreliable. Moreover, any disagreement between the sound speed model c₀ and the actual sound speed distribution c(r) produces significant aberrations in the ultrasound image. The resulting resolution and contrast are then insufficient for practical use of the image, particularly in medical imaging.

To address these issues, two methods are considered:
1) According to a first embodiment of the method for forming an ultrasound image, a first approach is used that consists in disassociating the depth z of the virtual transducers from the echo time t in order to align the focal plane with the isochronous volume. This alignment is obtained for each point of the image by optimising the confocal spot with respect to the parameter z which controls the curvature of the delay laws applied.
2) According to a second embodiment of the method for forming an ultrasound image, a second approach is used that consisting in varying (scanning) the sound speed model c₀. Alignment between the focal plane and the isochronous volume is obtained by optimising the confocal spot with respect to the sound speed model c₀.

### 1) Compensating for a Focusing Error/Focal Position Error

The first approach of the first embodiment of the method for ultrasonic characterisation of the medium is, for example, adapted to the following situation: imaging a medium whose tissues have a homogeneous sound speed c equal to the sound speed model (c=c₀) but whose ultrasound image is degraded by the presence of several layers of tissues with different sound speeds ct⁽¹⁾ upstream of these tissues; (i) is an index in a plurality of layers, and (l) stands for layer.

FIG. 8 illustrates the problem faced. The results of three experiments are shown. The first experiment in FIG. 8(a₁) is a reference experiment on an ultrasound phantom mimicking the acoustic properties of a medium whose effective sound speed is substantially homogeneous (c=1540 m/s). This phantom contains a random distribution of sub-resolved scatterers generating ultrasound speckle, as in soft tissue (agar-agar in our example), as well as several echogenic scatterers (nylon threads) and a cylinder with acoustic characteristics differing from the surrounding speckle (symbolised by the 5 points and the black disk in FIGS. 8(a₁) to 8(a₃)).

In the other two experiments (FIGS. 8(a₂) and 8(a₃)), a first aberrating layer is introduced between the transducer array (the probe) and the phantom (the medium).

In FIG. 8(a₂) this is a single layer of water with a uniform sound speed cₗ=1480 m/s and in FIG. 8(a₃) it is a single layer formed by a plexiglass plate with a uniform sound speed cₗ=2690 m/s.

Although the sound speed model c₀ coincides with the sound speed in the phantom in the present case, the first layer (water) degrades the resolution of the ultrasound image compared to the reference image, as evidenced by the impaired resolution of the bright points and the degraded speckle contrast in the corresponding ultrasound image in FIG. 8(b₂) compared to the reference image in FIG. 8(b₁). Due to a sound speed that is significantly different from that of the phantom, the plexiglass plate generates a stronger aberration in the ultrasound image and spurious echoes due to multiple reverberations, as shown in the corresponding ultrasound image in FIG. 8(b₃).

To understand the nature of the aberration induced by a layer with a different sound speed, we use the same analogy as in FIG. 6 between beamforming and numerical time reversal, and provide a new illustration of this reasoning in FIG. 9 for the case of a multi-layer (two-layer) medium.

A scatterer s at depth zₛ is associated with the echo time tₛ=2. [(zₛ−zₗ)/c₀+zₗ/ciₗ]

It therefore appears at depth z in the image in FIG. 9(c):

$${𝓏}_{t} = {c}_{0} {t}_{s} / 2 = {𝓏}_{s} - {𝓏}_{l} + {𝓏}_{l} {c}_{0} / {c}_{l} \qquad ( 5 )$$

The image of the scatterer is strongly aberrated due to the mismatch between the sound speed model used and the sound speed distribution upstream of the scatterer caused by the first aberrating layer cₗ. Under a paraxial approximation, it is known that the position of the focal plane is given by:

$${𝓏}_{t} = {𝓏}_{t} + Δ {𝓏}_{l} \qquad ( 6 )$$

$$with$$

$$Δ {𝓏}_{f} = ( \frac{{c}_{1}}{{c}_{0}} - \frac{{c}_{0}}{{c}_{1}} ) {z}_{l} \qquad ( 7 )$$

where zₗ is the thickness of the first aberrating layer.

The parameter Δz_{f} can be considered to be a focusing error, as illustrated in FIG. 9(c). Remarkably, this error does not depend on the position zₛ of the scatterer in the second layer of the medium if the sound speed model is correct and equal to c₀: c=c₀.

To correct this “focusing error,” or in other words the focal position error, the method of the present disclosure proposes to vary the focal depth z independently of the time-of-flight t, whereas these two parameters are generally intrinsically linked in a conventional beamforming process.

A de-scanned reflection matrix R′ can then be constructed from the focused reflection matrix R by rearranging the rows and columns to obtain a two-dimensional matrix, thus allowing easier and less costly matrix calculations, particularly in terms of computation time, which is an important advantage for real-time or near-real-time use of the system. Each column of the de-scanned reflection matrix corresponds to an element of a pair {x, t} and each row corresponds to an element of a pair {Δx, Δz}. This new de-scanned reflection matrix is therefore defined by:

$${R}^{′} = [ {R}^{′} ( \{ Δ x , Δ 𝓏 \} , \{ x , t \} ) ] .$$

In other words, the de-scanned reflection matrix R′ is expressed in a reference frame of the first point P1 of spatial position (xᵢₙ, zᵢₙ), where xᵢₙ=x and zᵢₙ=zₜ+Δz and with the second point P2 of spatial position (xₒᵤₜ zₒᵤₜ) referenced relative to the first point P1, i.e. xₒᵤₜ=x+Δx, zₒᵤₜ=zₜ++Δz. This expression can also be written in another reference frame, such as that of the second point P2, the midpoint between P1 and P2, or any other reference point, by a simple change of variables readily performed by a person skilled in the art.

This de-scanned reflection matrix R′ makes it possible to easily extract, for each point {x, t} in the medium, a local image of the focal spot in the dimensions {Δx, Δz}, where:
Δx=xₒᵤₜ−xᵢₙ is the lateral offset between the input and output transducers; and
Δz is the value of the focusing error between the focal depth and the expected depth zₜ of the isochronous volume for a medium with sound speed c₀.

Thus, the de-scanned reflection matrix R′ is constructed from the focused reflection matrix R by:

$${R}^{′} ( \{ Δ x , Δ 𝓏 \} , \{ x , t \} ) = R ( x + Δ x , x , t , {𝓏}_{t} + Δ , Δ 𝓏 ) \qquad ( 8 )$$

To simplify the expressions, a position offset may be introduced as a pair combining the lateral offset and the depth offset: Δr={Δx, Δz}.

The de-scanned reflection matrix R′ can also be constructed directly from the experimental reflection matrix Rᵤᵢ. In this case, the de-scanned reflection matrix includes the beamforming computation of the focused reflection matrix R, and this may be written as:

$${R}^{′} ( \{ Δ x , Δ 𝓏 \} , \{ x , t \} , {c}_{0} ) = \frac{1}{{N}_{in} {N}_{out}} {\sum }_{{i}_{in} , {u}_{out}} {R}_{ui} ( {u}_{out} , {i}_{in} , t + {τ}^{′} ( x , {i}_{in} , {𝓏}_{t} + Δ 𝓏 , {c}_{0} ) + τ ( x + Δ x , {u}_{out} , {𝓏}_{t} + Δ 𝓏 , {c}_{0} ) ) \qquad ( {8}^{′} )$$

In other words, in all cases, the method according to the first embodiment will include a step of determining a de-scanned reflection matrix R′,
which comprises, for a set of points in the medium of abscissa x and echo time t,
responses of the medium computed by beamforming from the experimental reflection matrix Rᵤᵢ(t), between an input virtual transducer of expected spatial position rᵢₙ=(x, zₜ+Δz) and an output virtual transducer of expected spatial position rₒᵤₜ=(x+Δx, zₜ+Δz), both virtual transducers being located at the same expected depth zₜΔz, where zₜ is the expected depth of the isochronous volume for a sound speed c₀, the isochronous volume being the set of points in the medium that contribute to the signals received at echo time t,
the responses of the de-scanned reflection matrix R′ being determined for a set of lateral offset values Δx including at least Δx=0 and for a set of depth offset values Δz,
said de-scanned reflection matrix R′ being formed such that each row corresponds to an element of a pair Δr={Δx, Δz}} which corresponds to a focal spot image, and each column corresponds to an element of a pair {x, t}, which is written as:

$${R}^{′} = [ {R}^{′} ( \{ Δ x , Δ 𝓏 \} , \{ x , t \} ) ] .$$

FIG. 10(a) shows an example of an image representing the apparent focal spot for a point {x, t} in the field of view which can be visualised from the above de-scanned reflection matrix. This image corresponds to a column of the de-scanned reflection matrix R′, shown as a two-dimensional image with the lateral offset Δx on the x-axis and the depth offset Δz on the y-axis.

The method further comprises:

An extraction step in which, from each focal spot image, an optimal position offset Δrₒₚₜ is determined close to the maximum in said focal spot image.

This optimal position offset Δrₒₚₜ={Δxₒₚₜ, ΔZₒₚₜ} substantially corresponds to the focusing error or a focal position error in the beamforming processes.

The maximum in the focal spot image is the point in this focal spot image that has the largest magnitude value relative to the entire focal spot. This means that this maximum corresponds to the point in the focal spot image with the highest magnitude. By “close to the maximum,” it is meant that a position close to the position of this maximum point is extracted. In practice, it is possible to take the closest possible point, or for example another point according to different criteria, such as a point having zero lateral offset (Δx=0), or another point between these two, or to use another criterion for the neighbourhood of the maximum point. The definition of the neighbourhood may, for example, be based on a desired spatial resolution.

This apparent focal spot, for example as shown in FIG. 10(a), is simultaneously modulated by the random reflectivity of the medium and contaminated by multiple-scattering echoes occurring upstream of the isochronous volume.

To overcome these speckle issues and obtain a more accurate estimator of the focal spot, and therefore of the focusing quality, averaging may be carried out over adjacent speckle grains. To do this, a spatio-temporal window of dimensions (Lₓ, Lₜ) is considered, over which the focal spot around each virtual transducer of spatial position rᵢₙ is averaged incoherently.

Thus, the method according to this first embodiment may optionally comprise, prior to the extraction step:

a step of spatio-temporal averaging of the de-scanned reflection matrix R′ in which the values of the de-scanned reflection matrix R′ are averaged locally along the lateral dimension x and along the echo time dimension t, for each element of a pair Δr={Δx, Δz}, the focal spot images thus being averaged.

The spatio-temporal averaging step therefore serves to average or smooth the focal spot image obtained from the de-scanned reflection matrix, in order to more correctly extract the position of the maximum in this image, this position corresponding to an offset to be used to correct the computation of the ultrasound image, as will be explained later.

This spatio-temporal averaging step may be implemented in various ways, using different weighted or unweighted averaging formulas, with or without normalisation.

According to a first variant of the spatio-temporal averaging, illustrated in FIG. 10(b), an incoherent point spread function PSF_{inc} is used, which can be calculated by:

$${PSF}_{inc} ( Δ r , \{ x , t \} ) = \sqrt{{〈 {\vert {R}^{′} ( Δ r , \{ {x}^{′} , {t}^{′} \} ) W ( {x}^{′} - x , {t}^{′} - t ) \vert}^{2} 〉}_{[ {x}^{′} , {t}^{′} ]}} \qquad ( 9 )$$

in which:
< > is an averaging operator that calculates an average for the parameters {x′, t′}
W(x′, t′) is a spatio-temporal weighting window.

The spatio-temporal weighting window may, for example, be a rectangular window, a Hanning window or a Gaussian window. A rectangular spatio-temporal weighting window can be defined by:

$$\begin{matrix}W ( {x}^{′} , {t}^{′} ) = 1 \vert {x}^{′} \vert < {L}_{x} / 2 and \vert t ’ \vert < {L}_{t} / 2 , and \\ W ( x ’ , t ’ ) = 0 otherwise\end{matrix} \qquad ( 10 )$$

The above incoherent point spread function PSF_{inc} provides, for a set of lateral offset values Δx including at least Δx=0 and for a set of depth offset values Δz, denoted as the pair Δr={Δx, Δz}, values corresponding to a focal spot image at lateral position x and echo time t. This focal spot image is smoothed with respect to speckle and multiple scattering effects, as shown in FIG. 10(b).

The depth position of the maximum of this focal spot image, Δzₒₚₜ, constitutes a first relevant estimator of the focusing error Δz_{f} sought in Equation 7.

In this case, the depth position of the maximum, Δzₒₚₜ, can be estimated by the position of the maximum of the magnitude of the point spread function PSF_{inc}.

More generally, the position of the maximum Δrₒₚₜ^{(inc)} can be estimated by the position of the maximum of the magnitude of the point spread function PSF_{inc}.

$$\begin{matrix}Δ {r}_{opt}^{( inc )} ( x , t ) - {arg max}_{Δ r} ( {PSF}_{inc} ( Δ r , \{ x , t \} ) ) \\ with Δ {r}_{opt}^{( inc )} = ( Δ {x}_{opt}^{( inc )} , Δ {𝓏}_{opt}^{( inc )} )\end{matrix} \qquad ( 11 )$$

Thus, the position offset of the maximum Δrₒₚₜ^{(inc)} can be extracted using the same formula (Equation 11), i.e. the position of the maximum in the focal spot image (the focal spot image after spatio-temporal averaging). This maximum position corresponds to the focusing error, or focal position error, arising in the beamforming process due to variations in sound speed within the medium, i.e., a medium presenting inhomogeneities in the sound speed in the different tissues constituting it.

In this way, during the extraction step, a maximum point of the focal spot image is sought, corresponding to or close to a maximum of the magnitude of the points in the focal spot image calculated using the incoherent point spread function, and the optimal position offset Δrₒₚₜ is the position of said maximum point in said focal spot image, or a position close to this maximum point, as explained previously.

It should be noted that in practice it is not always necessary to compute or prepare the complete image of the focal spot, and in particular it is often not necessary to scan points along the transverse direction Δx, since the maximum can be assumed to be located on the Δx=0 axis or very close to this axis. Indeed, to estimate the focusing error in the depth direction, Δz, it is sufficient to study the axial evolution of the confocal spot, that is to say for a set of points with Δx=0, i.e. only for spatial positions where xᵢₙ=xₒᵤₜ as illustrated by the first curve G1 in FIG. 10(g) which represents the evolution of the values of the focal spot image in FIG. 10(b) along the Δx=0 axis.

However, this estimator is biased by an incoherent background, evidenced by a relatively low contrast as can be seen in FIG. 10(b), and produces a broadening of the peak of the first curve G1 in FIG. 10(g).

According to a second variant of the spatio-temporal averaging, the confocal spot can be extracted coherently by intelligently recombining the apparent focal spots obtained on each window W. This allows a more accurate measurement of the depth offset Δz, and more generally of the position offset Δr.

This recombination is carried out in practice using a singular value decomposition (SVD) of a local matrix of focal spots,

$${R}^{( l )} ( \{ x , t \} ) = \vert {R}^{′} ( Δ r , \{ {x}^{′} , {t}^{′} \} ) W ( {x}^{′} - x , {t}^{′} - t ) ]$$

considered for each spatio-temporal window W(x, t), and expressed as:

$${R}^{( l )} ( x , t ) = {\sum }_{p} {λ}_{p} {U}_{p} ( \{ x , t \} ) {V}_{p}^{†} ( \{ x , t \} ) \qquad ( 12 )$$

in which the singular values λₚ are arranged, for example, in descending order.

Each singular value λₚ is associated with:
an output eigenvector Uₚ({x,t})=[Uₚ(Δr,{x,t})] defined in a de-scanned, focused frame (Δr), and
an input eigenvector Vₚ({x,t})=[Vₚ({x′,t′},{x,t})] defined in the conventional focused frame, i.e. the frame of the pixels of the ultrasound image.

Among these eigenvectors, the first eigenspace is of particular interest because:
(i) the coefficients of V₁({x, t}) directly give the phase shift to be applied to each speckle grain at {x′,t′} in order to rephase them;
(ii) the vector U₁({x, t}) provides the coherent confocal spot resulting from the in-phase recombination of each apparent focal spot measured around each point {x′, t′}:

A coherent point spread function PSF_{coh} can then be computed using:

$${PSF}_{coh} ( Δ r , \{ x , t \} ) = {U}_{1} ( Δ r , \{ x , t \} ) \qquad ( 13 )$$

Each coherent confocal spot PSF_{coh}(Δr, {x, t}) accurately quantifies the focusing process at each point {x, t} of the acoustic field. Owing to its coherent nature, this coherent point spread function PSF_{coh} makes it possible to obtain an image with much better contrast, as illustrated by the image in FIG. 10(c) (corresponding to the amplitude of the coherent point spread function PSF_{coh}) than an incoherent average of the confocal spots, as illustrated by the image in FIG. 10(b). The singular value decomposition (SVD) defined by Equation 12 enables each focal spot to be recombined by compensating the phase of each speckle grain at the point {x′, t′}. Echoes associated with scattering events outside the focal point {{x, t} are thereby drastically reduced.

As was done above using the incoherent point spread function PSF_{inc} (see Equation 12), a new estimator of the focusing error can be constructed by considering the value of the depth offset Δz that maximizes the amplitude of the coherent point spread function PSF_{coh} as illustrated by curve G2 in FIG. 10(g).

More generally, the position of the maximum Δrₒₚₜ^{(coh)} of the coherent point spread function is given by:

$$Δ {r}_{opt}^{( coh )} ( x , t ) = {arg max}_{Δ r} ( \vert {PSF}_{coh} ( Δ r , \{ x , t \} ) \vert ) \qquad ( 14 )$$

The uncertainty of the estimator

$$Δ {z}_{opt}^{( coh )}$$

is given for example (in the depth direction) by the ratio between the depth of field of the imaging system and the square root of the number of speckle grains Nₛ covered by the spatio-temporal window W, i.e.:

$$δ ( Δ {𝓏}_{opt}^{( coh )} c ) ∼ \frac{2 λ}{N {A}^{2} \sqrt{{N}_{s}}} \qquad ( 15 )$$

More generally, it is possible to use the same Equation 14 to extract the position offset of the maximum Δrₒₚₜ={Δxₒₚₜ, ΔZₒₚₜ}, i.e. the position of the maximum in the focal spot image (the focal spot image obtained by spatio-temporal averaging). This position of the maximum corresponds to the focusing error or focal position error occurring in the beamforming process due to inhomogeneities in the sound speed within the medium.

In this way, during the extraction step, a maximum point of the focal spot image is sought, corresponding to or close to a maximum of the magnitude of the points of the focal spot image calculated by the coherent point spread function, and the optimal position offset Δrₒₚₜ is the position of said maximum point in said focal spot image, or a position close to this maximum point, as explained previously.

According to a third variant of the spatio-temporal averaging, it is also possible to use the information contained in the phase of the coherent confocal spot, as illustrated in FIG. 10(d), for example by examining the imaginary part of the coherent point spread function PSF_{coh}. This provides a more accurate estimator of the depth offset Δz, and more generally of the position offset Δr.

The phase of the confocal spot presents a π phase shift at the focal plane, as illustrated in FIG. 10(e), which shows the phase of the coherent point spread function PSF_{coh} along the Δx=0 axis. The coherent confocal spot accumulates the Gouy phase shifts (~π/2 in a 2D configuration) induced by the focusing processes on transmission and reception. These Gouy phase shifts occur at the depth z+Δz_{f} where the transverse confinement of the focused waves is minimal. It therefore constitutes a more reliable estimator of the focusing error than the magnitude of the coherent point spread function PSF_{coh} or of the incoherent point spread function PSF_{inc}, as can be seen from curve G3 in FIG. 10(g), which are also sensitive to the geometric attenuation of the focused wave. To increase the sensitivity of the method, it is also possible to combine the two previous quantities, the magnitude and the phase of the coherent point spread function PSF_{coh}, by examining the imaginary part of this function (see the curve in FIG. 10(f)), which shows the imaginary part of the coherent point spread function PSF_{coh} along the Δx=0 axis). Its inflection point, i.e. the maximum of its axial derivative or derivative along the depth direction Δz, provides a new estimator of the optimal depth offset ΔZₒₚₜ (see curve G4 in FIG. 10(g)).

This estimator can be generalised by searching for the maximum of the magnitude of the complex derivative of the point spread function PSF_{coh}:

$$Δ {r}_{opt}^{( gouy )} ( x , t ) = {arg max}_{Δ r} ( \vert {\partial }_{Δ 𝓏} {PSF}_{coh} ( Δ r , \{ x , t \} ) \vert ) \qquad ( 16 )$$

According to an uncertainty calculation, this estimator significantly improves the determination of Δz by a factor of \sqrt{6}, i.e. approximately 2.5, compared to Δ\hat{z}^{(coh)} in Equation 15.

FIG. 8(c) shows the histogram of the estimated focusing errors ΔZₒₚₜ for the three experiments described in FIG. 8(a). While the reference experiment yields an estimate of the focusing error close to zero (0), ΔZₒₚₜ is close to its expected value both for the water layer (ΔZₒₚₜ=−1.4 mm) and for the plexiglass plate (Δzₒₚₜ=+8.5 mm).

The dispersion of the estimated focusing error values highlighted in FIG. 8(c) is related to the paraxial approximation used to establish Equation 7, which is not strictly verified in the experiments in FIG. 8(a).

More generally, the position offset of the maximum Δrₒₚₜ={Δxₒₚₜ, ΔZₒₚₜ} can be extracted using the same Equation 14, i.e. the position of the maximum in the focal spot image (the focal spot image obtained by spatio-temporal averaging). This position of the maximum corresponds to the focusing error or focal position error occurring in the beamforming process due to variation of the sound speed within the medium.

In this way, during the extraction step, a maximum point of the focal spot image is sought, corresponding to:
a maximum of the magnitude of the points (values) of the focal spot image, or
a maximum of the magnitude of the derivative, along the depth direction (axial direction), of the points (values) of the focal spot image, or
a maximum of the magnitude of the derivative, along the depth direction (axial direction), of the imaginary part of the points (values) of the focal spot image.

The optimal position offset Δrₒₚₜ is the position of the maximum point in said focal spot image, or a position close to this maximum point, as explained previously.

The optimal position offset Δrₒₚₜ or the optimal depth offset ΔZₒₚₜ, can be used to obtain a new ultrasound image that is correctly focused, i.e. with a correction of the focal position error:
either from the focused reflection matrix R:

$${ℐ}_{1} ( x , t ) = \vert R ( x + Δ {x}_{opt} ( x , t ) , x , t , {z}_{t} + Δ {𝓏}_{opt} ( x , t ) ) \vert \qquad ( 17 )$$

or directly from the de-scanned reflection matrix R′:

$${ℐ}_{1} ( x , t ) = \vert {R}^{′} ( Δ r = Δ {r}_{opt} ( x , t ) , \{ x , t \} ) \vert \qquad ( 18 )$$

Or, if Δxₒₚₜ is considered equal to 0 or negligible, by:

$${ℐ}_{1} ( x , t ) = \vert {R}^{′} ( \{ Δ x = 0 , Δ 𝓏 = Δ {z}_{opt} ( x , t ) \} , \{ x , t \} ) \vert$$

The optimal position offset Δrₒₚₜ can advantageously be used with both of its components, as expressed in Equation 17 above.

Thus, the method according to the first embodiment may additionally comprise:
a step of forming an ultrasound image by computing the reflectivity at a plurality of points in the medium, from the de-scanned reflection matrix R′, the reflectivity of each of these points in the medium corresponding to the response of the medium between the input virtual transducer at spatial position rᵢₙ=(x, zₜ++Δzₒₚₜ) and the output virtual transducer at spatial position rₒᵤₜ=r+Δrₒₚₜ=(x+Δxₒₚₜ, zₜ+Δzₒₚₜ), and said reflectivity being defined by:

$${ℐ}_{1} ( x , t ) = \vert {R}^{′} ( \{ Δ r = Δ {r}_{opt} ( x , t ) , \{ x , t \} ) \vert .$$

The ultrasound image presented above is defined in terms of the lateral position x and the echo time t.

This ultrasound image can be transformed so as to be defined in terms of the lateral position x and the depth z by using the sound speed model c₀ to replace the echo time t. This transformation consists of a change of variables or parameters. The resulting image is identical, but with dimensions given in distance units as commonly used, allowing the clinician to perform measurements of structure sizes. The ultrasound image is then of the following type:

$${\tilde{ℐ}}_{1} ( x , {𝓏}_{t} ) = {ℐ}_{1} ( x , t = 2 {𝓏}_{t} / {c}_{0} ) ,$$

in which zₜ is the depth of a pixel of the ultrasound image, or the depth of the isochronous volume.

Either of the above ultrasound images can be displayed on the display device 43.

According to one variant, a video is formed from a sequence of ultrasound images produced over a first number N of iterations of the steps of the method. The method is thus iterated to form ultrasound images at a predetermined rate, enabling substantially real-time viewing of the evolution of the ultrasound image of the medium.

According to one variant:
among the N iterations, a second number P (smaller than N) of iterations do not include the steps of determining the de-scanned reflection matrix R′, the spatio-temporal averaging steps, or the steps of extracting an optimal position offset, and
for these P (specific) iterations, in the ultrasound image formation step, the ultrasound image is obtained from an optimal position offset extracted during a previous iteration.

Thus, for a number P of iterations, the calculations performed in the steps of determining the de-scanned reflection matrix R′, the spatio-temporal averaging steps and the steps of extracting an optimal position offset, are advantageously avoided. The method according to this variant requires fewer computational and/or time resources while still providing a video sequence consisting of high-quality ultrasound images. Thanks to this arrangement, the rate of ultrasound image generation can be decoupled from the rate of calculation of the optimal position offset. Consequently, a high ultrasound image frame rate can be maintained despite a reduced number of calculations.

Furthermore, the previous calculations of the method according to the present description may be carried out only in the axial direction (the depth z direction). Thus, in the extraction step, an optimal position offset Δrₒₚₜ={Δxₒₚₜ, ΔZₒₚₜ} is determined. In the present case, it is possible to restrict the determination to only the second component of the optimal position offset, i.e. the depth offset ΔZₒₚₜ. Thus, in this variant, the first component of the optimal position offset, i.e. the lateral offset Δxₒₚₜ, is considered to be zero, Δxₒₚₜ=0. This simplifies the computation of the ultrasound image and reduces its overall cost in terms of time and resources.

Similarly, in the step of determining a de-scanned reflection matrix R′, the de-scanned reflection matrix R′ may be formed only for lateral offset values Δx equal to zero, Δx=0. The need to compute a complete de-scanned reflection matrix is thus avoided, which greatly reduces the overall time and resource cost of computing this de-scanned reflection matrix, and also reduces the cost of computing the subsequent steps of the method after this step of determining the de-scanned reflection matrix.

Optionally, in the spatio-temporal averaging step, the averaging of the values of the de-scanned reflection matrix R′ may be limited along the lateral dimension x and along the echo time dimension t, to the elements of the pairs {Δx=0, Δz} of the focal spot images whose lateral offset Δx is zero (Δx=0). The number of averaged focal spot images to be computed is thus reduced, thereby reducing the computation cost.

Furthermore, in the extraction step, the optimal depth offset ΔZₒₚₜ can be determined from the points or values of the focal spot image that have a lateral offset Δx equal to zero, Δx=0. Thus, the optimal lateral offset Δxₒₚₜ is zero, and the optimal depth offset Δzₒₚₜ has a value determined solely from the points of a curve of the focal spot image corresponding to zero lateral offset. The spatial offset position determined, Δrₒₚₜ, is therefore slightly different from the position of the maximum of the focal image, but remains close to it, since the lateral offset must intrinsically be small. The computation cost of this variant is thus advantageously greatly reduced.

The ultrasound images corresponding to the results of the method described herein, which correct the focusing error or focal plane position error, are shown in FIG. 8(d). In the case of the first water layer, the corrected ultrasound image is shown in FIG. 8(d₂). This ultrasound image has the same resolution as the reference image in FIG. 8(d₁), i.e. the ultrasound image obtained without this first aberrating layer. Applying a single correction to the curvature of the focusing laws (which is what the present correction does) corrects the whole of the ultrasound image. The same effect is observed in the case of the first layer consisting of a plexiglass plate, in FIG. 8(d3). This result is striking when this corrected ultrasound image is compared with the initial image in FIG. 8(b₃). It should be noted, however, that the present correction of the focusing error does not entirely compensate for the reverberations induced by the first aberrating layer.

The corrected ultrasound image results in FIG. 8 are interesting from an academic point of view, but nevertheless remain limited because these cases consider only a single aberrating layer that is translation-invariant. FIG. 11 shows an in vivo ultrasound imaging experiment on a liver in which an irregular natural arrangement of adipose and muscular tissues, and other vessels, impairs the ultrasound image quality (see FIG. 11(a)). The method described above is extended to the case of a curved probe, which amounts to replacing the Cartesian coordinates (x, z) with polar coordinates (θ, r). FIG. 11(b) shows a map of the estimated focusing error at each point in the image, i.e. a map of an axial position offset Δr computed using the method. In contrast to the previous experiments in FIG. 8, this focusing error is not homogeneous across the field of view. This is notably due to the lateral variations (Δθ in polar coordinates) in the sound speed within the aberrating layers, which result in a substantial lateral variation in the focusing error. The axial variation of the focusing error in the liver is related to the mismatch between the sound speed model c₀ and the effective sound speed c in the liver.

Finally, the above method may be supplemented to further determine an integrated sound speed c̅opt from the optimal depth offset Δzₒₚₜ and the optimal position offset Δrₒₚₜ={Δxₒₚₜ, ΔZₒₚₜ}, by:

$${\overline{c}}_{opt} ( x , t ) = \frac{{c}_{0}}{\sqrt{1 + \frac{2 Δ {𝓏}_{opt} ( x , t )}{{c}_{0} t}}} .$$

This integrated sound speed corresponds to an average sound speed between the transducer array and the depth of the isochronous volume. This integrated sound speed can be determined for any pair {x, t}, that is to say, for any focal spot image, i.e. for any point in the medium or any point in an ultrasound image.

The local sound speeds c(r) can then be computed for a set of pairs {x, t}, from integrated sound speed values c̅opt obtained for the focal spot images of the pairs {x, t}. This computation, which makes it possible to derive local sound speeds from an integrated sound speed field, is generally performed by an inversion method. An example of an inversion method of this type will be described in more detail later, at the end of the description of the second embodiment of the method, which is specifically devoted to the more accurate determination of a sound speed model (integrated sound speed model).

The method may then further comprise a step of forming a local sound speed image of the medium from the set of previously determined integrated sound speed values, and a step of displaying the local sound speed image on the display device 43. This local sound speed image is important for the clinician because, when combined with other information, it is useful for identifying certain pathological conditions or diseases such as hepatic steatosis.

Having obtained a local sound speed field, it is then possible to iterate the method described above in order to obtain a more accurate ultrasonic characterisation of the medium, by recalculating a de-scanned reflection matrix R′ using the local sound speed values determined in a previous step.

As described above for this first embodiment of the method, each pixel of the ultrasound image can be corrected using the locally estimated focusing error (see Equation 17). The result is shown in FIG. 12. The corrected image in FIG. 12(b) advantageously shows an improvement in contrast and resolution compared to the initial image in FIG. 12(a). Veins that are difficult to make out in the initial image are clearly revealed after local correction of the focusing error on all or some of the image pixels. The correction of transverse aberrations is also quantified in FIG. 13, which shows, by way of illustration, an example of focused reflection matrices before and after correction. Whereas the backscattered energy initially extends well beyond the diagonal of the focused reflection matrix Rₓₓ shown in FIG. 13(b), compensating for the focusing error “brings back” most of the backscattered energy onto the diagonal of the focused reflection matrix Rₓₓ, as illustrated in its representation in FIG. 13(d). This is confirmed by examining the transverse spread of the incoherent point spread function PSF_{inc} before and after compensation of the focusing error, as illustrated by the curves in FIG. 13(e).

The method presented is particularly effective for compensating for axial aberrations (in the depth direction) in an ultrasound image. However, it does not fully allow the scatterers to be relocated exactly at their actual depth; the axial direction of the image remains governed by the time-of-flight t of the echoes, such that zₜ=c₀t/2. To this end, the following section proposes, instead of scanning the curvature of the focusing laws to determine a focusing error, to scan the sound speed model directly in order to map it and thus reposition each scatterer at its actual depth in the ultrasound image.

### 2) Sound-Speed Tomography

The second approach of the second embodiment of the method for ultrasonic characterisation of a medium consists in varying the sound speed c₀ of the sound speed model. A focused reflection matrix R_{xx,t}(c₀) is then constructed at the ballistic depth zₜ for the different sound speed models c₀. The procedure is therefore similar to that of the first approach, with variation of the sound speed model parameter c₀, and a person skilled in the art will be able to revise and adapt the calculation methods.

In a similar way to Equation 1, this focused reflection matrix can be expressed without the depth parameter z, since the depth is now adjusted to the expected depth of the isochronous volume zₜ:

$$R ( {x}_{out} , {x}_{in} , t , {c}_{0} ) = \frac{1}{{N}_{in} {N}_{out}} {\sum }_{{i}_{in} {u}_{out}} {R}_{ui} ( {u}_{out} , {i}_{in} , t + {τ}^{′} ( {x}_{in} , {i}_{in} , {z}_{t} , {c}_{0} ) + τ ( {x}_{out} , {u}_{out} , {z}_{t} , {c}_{0} ) ) \qquad ( 18 )$$

A de-scanned reflection matrix R′ can thus be constructed from the focused reflection matrix R by rearranging the rows and columns of this matrix to obtain a two-dimensional matrix for easier matrix calculations. Each column of the de-scanned reflection matrix corresponds to an element of a pair {x, t} and each row corresponds to an element of a pair {Δx, c₀}. Each row of the de-scanned reflection matrix therefore corresponds to a focal spot image. This new de-scanned reflection matrix R′ is therefore defined by:

$${R}^{′} = [ {R}^{′} ( \{ Δ x , {c}_{0} \} , \{ x , t \} ) ] \qquad ( 19 )$$

In other words, this de-scanned reflection matrix R′ is expressed in a reference frame based on the first point P1 of spatial position (xᵢₙ, zᵢₙ) where xᵢₙ=x and in relation to the second point P2 of spatial position=(xₒᵤₜ, zₒᵤₜ), with xₒᵤₜ=x+Δx. We also have zᵢₙ=zₒᵤₜ=zₜ. This expression can also be written in another reference frame, such as that of the second point P2, the midpoint or any other reference point, by a simple change of variable.

This de-scanned reflection matrix R′ makes it easy to extract, for each point {x, t} in the medium, a local image of the focal spot in the dimensions {Δx, c₀}, where:
Δr=xₒᵤₜ−xᵢₙ, is the lateral offset between the input and output transducers; and
c₀ is the value of the sound speed model.

Thus, the de-scanned reflection matrix R′ is constructed from the focused reflection matrix R (using Equation 18) by:

$${R}^{′} ( \{ Δ x , {c}_{0} \} , \{ x , t \} ) = R ( x + Δ x , x , t , {c}_{0} )$$

This de-scanned reflection matrix R′ contains a set of apparent focal spots for each speckle grain {x, t}.

The de-scanned reflection matrix R′ can also be constructed directly from the experimental reflection matrix Rᵤᵢ. Thus, the de-scanned reflection matrix incorporates the beamforming computation of the focused reflection matrix R, and can be written as:

$${R}^{′} ( \{ Δ x , {c}_{0} \} , \{ x , t \} ) = \frac{1}{{N}_{in} {N}_{out}} {\sum }_{{i}_{in} {u}_{out}} {R}_{ui} ( {u}_{out} , {i}_{in} , t + {τ}^{′} ( x , {i}_{in} , {z}_{t} , {c}_{0} ) + τ ( x + Δ x , {u}_{out} , {𝓏}_{t} , {c}_{0} ) )$$

In other words, the method according to this second embodiment will comprise a step of determining a de-scanned reflection matrix R′,
which comprises, for a set of points of the medium of abscissa x and echo time t,
responses of the medium calculated by beamforming according to a sound speed model c⁰ from the experimental reflection matrix Rᵤᵢ(t), between an input virtual transducer of spatial position rᵢₙ=(x, zₜ) and an output virtual transducer of spatial position rₒᵤₜ=(x+Δx, zₜ), both virtual transducers being at the same depth zₜ, zₜ being the depth of the isochronous volume expected for the sound speed c₀, the isochronous volume being the set of points of the medium contributing to the signals received at time t,
the responses of the de-scanned reflection matrix R′ being determined for a set of lateral offset values Δx including at least Δx=0 and for a set of sound speed models c₀,
said de-scanned reflection matrix being formed such that each row corresponds to an element of a pair {Δx, c₀} which corresponds to a focal spot image, and each column corresponds to an element of a pair {x, t}, which is written as:

$${R}^{′} = [ {R}^{′} ( \{ Δ x , {c}_{0} \} , \{ x , t \} ) ]$$

This de-scanned reflection matrix R′ makes it possible to obtain an image representing the apparent focal spot for a point {x, t} in the field of view. This focal spot image corresponds to a column of the de-scanned reflection matrix R′, shown as a two-dimensional image with the lateral offset Δx and the sound-speed model c₀ on the y-axis.

The method further comprises:
an extraction step, in which an optimal lateral offset Δxₒₚₜ and an optimal sound speed model c̅ₒₚₜ close to the maximum in the focal spot image are determined from each focal spot.

The optimal sound speed model c̅ₒₚₜ then corresponds to a sound speed model close to the sound speed distribution in the medium being examined.

This optimal sound speed model is advantageously much closer to the (actual) sound speed of the medium than the commonly used assumption of a constant sound speed in the medium.

The maximum in the focal spot image is the point in this focal spot image that has the largest magnitude value relative to the entire focal spot. This means that this maximum corresponds to the point in the focal spot image with the highest magnitude. By “close to the maximum,” it is meant that a point close to this maximum point is extracted. In practice, it is possible to take the closest possible point, or for example another point according to different criteria, such as a point having zero lateral offset (Δx=0), or another point between these two, or to use another criterion for the neighbourhood of the maximum point. The definition of the neighbourhood may, for example, be based on a desired spatial resolution and/or a desired sound speed resolution.

As in the first embodiment of the method, this apparent focal spot is modulated because of the ultrasound speckle issue and multiple scattering. A statistical average of these focal spots is calculated in a similar way over a spatio-temporal window of dimensions (Lₓ, Lₜ).

Thus, the method according to the second embodiment also comprises:
a step of spatio-temporal averaging of the de-scanned reflection matrix R′ in which the values of the de-scanned reflection matrix are averaged locally along the lateral dimension x the dimension of the time echo t, for each element of a pair {Δx, c₀}, the resulting focal spot images then being averaged.

This spatio-temporal averaging step makes it possible to correctly average or smooth the focal spot image obtained from the de-scanned reflection matrix, in order to more accurately extract the position of the maximum in this image, a position that corresponds to the lateral offset and the sound speed model to be used to correct the computation of the ultrasound image.

This spatio-temporal averaging step may be implemented in various ways, using different weighted or unweighted averaging formulas, with or without normalisation.

According to a first variant of the spatio-temporal averaging, the intensity of the de-scanned reflection matrix is averaged locally by calculating an incoherent point spread function PSFᵢₙₑ on input virtual transducers {x′, t′} around a point of interest {x, t}, for example by the following expression:

$${PSF}_{inc} ( \{ Δ x , {c}_{0} \} , \{ x , t \} ) = \sqrt{{( {\vert {R}^{′} ( \{ Δ x , {c}_{0} \} , \{ {x}^{′} , {t}^{′} \} ) W ( {x}^{′} - x , {t}^{′} - t ) \vert}^{2} )}_{[ {x}^{′} , {t}^{′} ]}} \qquad ( 20 )$$

in which:
<.> is an averaging operator with respect to the parameters of the pair {x′, t′}
R′ is the de-scanned reflection matrix,
W(x′, t′) is a spatio-temporal weighting window using the same parameters.

The spatio-temporal weighting window may be of any type as defined in the description of the first embodiment.

This incoherent point spread function provides, for a set of lateral offset values Δx including at least Δx=0 and for a set of sound speed model values c₀, values corresponding to a focal spot image for the lateral position x and the echo time t.

The incoherent point spread function PSFᵢₙₑ obtained from the data of the k-Wave simulation shown in FIG. 7 for the first embodiment of the method is now illustrated for the second embodiment in FIG. 14(a).

The position of the maximum of the incoherent point spread function PSF_{inc}, defined by PSF_{inc}({Δx, c₀}, {x, t}), provides, on its y-axis, an estimator of the integrated sound speed c̅, i.e.

$${\overline{c}}_{opt}^{( inc )} ( x , t ) ,$$

at each point {x, t} of the focal spot image. The position of this maximum on the x-axis gives an optimal lateral offset. FIG. 14(d) shows a curve D1 illustrating the evolution of the incoherent point spread function for a zero lateral offset, i.e. Δx=0; in other words, the evolution of the point spread function on the Δx=0 axis.

It should be noted that in practice, as for the first embodiment, it is not always necessary to compute or prepare the complete image of the focal spot, and it is often not necessary to scan points along the lateral/transverse direction Δx, since the maximum can be assumed to be located on the Δx=0 axis. One may simply examine the axial evolution of the confocal spot, i.e. for Δx=0, which is equivalent to xᵢₙ=xₒᵤₜ, in order to estimate the integrated sound speed c̅ as shown by curve D1 in FIG. 14(d) which represents the evolution of the focal spot values on the Δx=0 axis.

In this first variant, in the extraction step, a maximum point of the focal spot image is sought, corresponding to a maximum of the magnitude of the points of the focal spot image, and the optimal sound speed model c̅ₒₚₜ is the ordinate of said maximum point in said focal spot image. The abscissa of the maximum point in the focal spot image is then the optimal lateral offset Δxₒₚₜ, in the same way as in the first embodiment.

The coordinates of the maximum point in the focal spot image are therefore obtained by:

$$\{ {\overline{c}}_{opt}^{( inc )} ( x , t ) , Δ {x}_{opt}^{( inc )} ( x , t ) \} = {arg max}_{\{ {c}_{0} , Δ x \}} ( {PSF}_{inc} ( \{ Δ x , {c}_{0} \} , \{ x , t \} ) ) \qquad ( 21 )$$

From this Equation 21, we can extract the optimal sound speed model c̅ₒₚₜ, which corresponds to the ordinate of the maximum point in the focal spot image produced (the focal spot image obtained by spatio-temporal averaging).

In this way, during the extraction step, a maximum point of the focal spot image is sought, corresponding to a maximum of the magnitude of the points of the focal spot image, and the optimal sound speed model c̅ₒₚₜ is the ordinate of said maximum point in said focal spot image.

Optionally, it is possible to extract the abscissa and ordinate of the maximum point in the focal spot image, i.e. respectively the optimal lateral offset Δxₒₚₜ and the optimal sound speed model c̅ₒₚₜ.

According to a second variant of the spatio-temporal averaging, the intensity of the de-scanned reflection matrix R′ is locally averaged by calculating a coherent point-spread function PSF_{coh}. This provides a better estimate of the sound speed model, which is an integrated sound speed model c̅.

For this purpose, as in the first embodiment, a singular value decomposition (SVD) of a local matrix of focal spots is performed, using the following expression:

$${R}^{( l )} ( \{ x , t \} ) = [ {R}^{′} ( \{ Δ x , {c}_{0} \} , \{ {x}^{′} , {t}^{′} \} ) W ( {x}^{′} - x , {t}^{′} - t ) ] ,$$

and
an expression similar to the previous Equation 13 is obtained.

The output eigenvectors are then:

$${U}_{p} ( \{ x , t \} ) = [ {U}_{p} ( \{ Δ x , {c}_{0} \} , \{ x , t \} ) ]$$

The first output eigenvector, U₁({x, t})=[U₁({Δx, c₀}, {x, t})], directly gives the evolution of the coherent focal spot with respect to the sound speed model c₀ around each point {x, t}, i.e.:

$${PSF}_{coh} ( \{ Δ x , {c}_{0} \} , \{ x , t \} ) = {U}_{1} ( \{ Δ x , {c}_{0} \} , \{ x , t \} ) \qquad ( 22 )$$

A new estimator of the integrated sound speed c̅ can be derived from the maximum of the coherent point spread function, i.e. by:

$$\{ {\overline{c}}_{opt}^{( coh )} ( x , t ) , Δ {x}_{opt}^{( coh )} ( x , t ) \} = {arg max}_{\{ {c}_{0} , Δ x \}} ( \vert {PSF}_{coh} ( \{ Δ x , {c}_{0} \} , \{ x , t \} ) \vert ) \qquad ( 23 )$$

More generally, the optimal sound speed model can be extracted using Equation 23, based on the position of the maximum in the focal spot image (the focal spot image obtained by spatio-temporal averaging).

In this way, during the extraction step, a maximum point of the focal spot image is sought, corresponding to a maximum of the magnitude of the points of the focal spot image, and the optimal sound speed model c̅ₒₚₜ is the ordinate of said maximum point in said focal spot image. The abscissa of the maximum point in the focal spot image is then the optimal lateral offset Δxₒₚₜ.

The focal spot image obtained via the coherent point spread function PSF_{coh} is shown in FIG. 14(b). This image advantageously offers better sensitivity than that obtained using the incoherent point spread function in FIG. 14(a).

Indeed, the accuracy of this estimator of the integrated sound speed c̅ is inversely proportional to the second derivative of the point spread function PSF around its maximum with respect to c₀, i.e.:

$$δ {\overline{c}}_{opt} ( x , t ) ∝ \frac{PSF ( \{ Δ x = 0 , {c}_{0} = {\overline{c}}_{opt} ( x , t ) \} , \{ x , t \} )}{{\partial }_{{c}_{0}}^{2} PSF ( \{ Δ x = 0 , {c}_{0} = {\overline{c}}_{opt} ( x , t ) \} , \{ x , t \} )} \qquad ( 24 )$$

FIG. 14(d) shows the values of the focal spot images for a zero lateral offset, i.e. Δx=0, in FIGS. 14(a) to 14(c). In particular, curve D1 in FIG. 14(d) corresponds to the evolution of the values of the incoherent point spread function PSF_{inc} at zero lateral offset in FIG. 14(a). Curve D2 corresponds to the evolution of the values of the coherent point spread function PSF_{coh} at zero lateral offset in FIG. 14(b). We note the sharper or narrower shape of the latter coherent confocal spot curve around its maximum. In other words, curve D2 presents a greater curvature at its maximum point and a higher signal-to-noise ratio, which is indicative of improved accuracy in estimating the integrated sound speed with this estimation variant:

$${\overline{c}}_{opt}^{( coh )} ( x , t ) .$$

According to a third variant of the spatio-temporal averaging, the axial derivative of the coherent point spread function PSF_{coh} PSF_{coh}, as shown in FIG. 14(c), is considered. This makes it possible to obtain an even more reliable estimator of the integrated sound speed c̅. The double Gouy phase shift at the focal plane is exploited. For this purpose, a new estimator of the integrated sound speed c̅ can be constructed from the maximum of the axial derivative of the coherent point spread function PSF_{coh}, i.e. by:

$$\{ {\overline{c}}_{opt}^{( gouy )} ( x , t ) , Δ {x}_{opt}^{( gouy )} ( x , t ) \} = {arg max}_{\{ {c}_{0} , Δ x \}} ( \vert {\partial }_{{c}_{0}} {PSF}_{coh} ( \{ Δ x , {c}_{0} \} , \{ x , t \} ) \vert ) \qquad ( 25 )$$

This estimator provides an excellent estimate of the position of the focal plane, as illustrated by curve D3 in FIG. 14(d). The second derivative of this parameter around its maximum is indeed much greater than that of the magnitude of the coherent point spread function PSF_{coh} and the magnitude of the incoherent point spread function PSF_{inc}.

More generally, the optimal sound speed model can advantageously be extracted using Equation 25, based on the position of the maximum in the focal spot image (the focal spot image obtained by spatio-temporal averaging).

In this way, during the step of extracting an optimal sound speed model, a maximum point of the focal spot image is sought, corresponding to:
a maximum of the magnitude of the points (values) of the focal spot image, or
a maximum of the magnitude of the derivative with respect to the sound speed c₀ of the points (values) of the focal spot image, or
a maximum of the magnitude of the derivative with respect to the sound speed c₀ of the imaginary part of the points (values) of the focal spot image.

The optimal sound speed model c̅ₒₚₜ is then the ordinate of said maximum point in said focal spot image.

The value of the optimal (integrated) sound speed model can be used to obtain a new corrected ultrasound image, i.e. one with a corrected sound speed model:
either from the focused reflection matrix R:

$${ℐ}_{2} ( x , t ) = \vert R ( x , x , t , {c}_{0} = {\overline{c}}_{opt} ( x , t ) ) \vert$$

or from the de-scanned reflection matrix R′:

$${ℐ}_{2} ( x , t ) = \vert {R}^{′} ( \{ Δ x = 0 , {c}_{0} = {\overline{c}}_{opt} ( x , t ) \} , \{ x , t \} ) \vert$$

Optionally, it is possible to use both coordinates (abscissa and ordinate), i.e. the optimal lateral offset Δxₒₚₜ and the optimal sound speed model c̅ₒₚₜ. The ultrasound image is then obtained:
either from the focused reflection matrix R:

$${ℐ}_{2} ( x , t ) = \vert R ( x + Δ {x}_{opt} ( x , t ) , x , t , {c}_{0} = {\overline{c}}_{opt} ( x , t ) ) \vert$$

or from the de-scanned reflection matrix R′:

$${ℐ}_{2} ( x , t ) = \vert {R}^{′} ( \{ Δ x = Δ {x}_{opt} ( x , t ) , x , t , {c}_{0} = {\overline{c}}_{opt} ( x , t ) ) \vert$$

Thus, the method of the present disclosure also comprises:
a step of forming an ultrasound image by computing the reflectivity at a plurality of points in the medium, from the de-scanned reflection matrix R′, the reflectivity of each of these points in the medium corresponding to the response of the medium between the input virtual transducer at spatial position rᵢₙ=(x, zₜ) and the output virtual transducer at spatial position rₒᵤₜ=(x+Δxₒₚₜ, zₜ), zₜ being the depth of the isochronous volume expected for the optimal sound speed model c̅ₒₚₜ and said reflectivity being defined by:

$${ℐ}_{2} ( x , t ) = \vert {R}^{′} ( \{ Δ x = Δ {x}_{opt} ( x , t ) , {c}_{0} = {\overline{c}}_{opt} ( x , t ) \} , \{ x , t \} ) \vert .$$

The ultrasound image above is defined in terms of the lateral position x and the echo time t.

This ultrasound image can be transformed so as to be defined in terms of the lateral position x and the depth z by using the sound speed model c̅ₒₚₜ to replace the echo time t. This transformation consists of a change of variables or parameters. The resulting image is identical, but with dimensions given in distance units as commonly used, allowing the clinician to perform measurements of structure sizes. The ultrasound image is then of the following type:

$${\tilde{ℐ}}_{2} ( x , \tilde{z} ) = {I}_{2} ( x , t = 2 \tilde{z} / {\overline{c}}_{opt} ( x , t ) ) \qquad ( 26 )$$

in which z is the depth of a pixel in the ultrasound image and the estimated depth of the isochronous volume.

Either of the above ultrasound images can be displayed on the display device 43.

According to one variant, a video is formed from a sequence of ultrasound images produced over a first number N of iterations of the steps of the method. The method is thus iterated to form ultrasound images at a predetermined rate, enabling substantially real-time viewing of the evolution of the ultrasound image of the medium.

According to one variant:
among the N iterations, a second number P (smaller than N) of iterations do not include the steps of determining the de-scanned reflection matrix R′, the spatio-temporal averaging steps, or the steps of extracting an optimal sound speed model, and
for these P (specific) iterations, in the ultrasound image formation step, the ultrasound image is obtained from an optimal sound speed model extracted during a previous iteration.

Thus, for a number P of iterations, the calculations performed in the steps of determining the de-scanned reflection matrix R′, the spatio-temporal averaging steps and the steps of extracting an optimal sound speed model are avoided. The method according to this variant requires fewer computational resources while still providing a video sequence consisting of high-quality ultrasound images. Thanks to this arrangement, the rate of ultrasound image generation can be decoupled from the rate of calculation of the optimal position offset. Consequently, a high ultrasound image frame rate can be maintained with a drastically reduced number of calculations.

The different variants for reducing the computation cost in the first embodiment can also be used and/or adapted to the second embodiment, concerning the search for an optimal sound speed model.

Specifically:
in the extraction step, the lateral offset Δxₒₚₜ can be considered to be zero, and/or
in the step of determining the de-scanned reflection matrix R′, it is possible to calculate only the values for a zero lateral offset Δx; and/or
in the spatio-temporal averaging step, the focal spot images corresponding to the pairs {Δx=0, Δz} are averaged, and/or
in the extraction step, the optimal sound speed model will be determined from the points of the focal spot image with a lateral offset 4x equal to zero.

These variants present advantages similar or even identical to those outlined in the first embodiment and allow computation to be reduced at various stages of the process.

All the optimal sound speed model values c̅ₒₚₜ can be assembled to form an integrated sound speed image. This integrated sound speed image can be displayed on the display device 43. However, it is preferable to calculate the local sound speed values c(r) as explained later.

FIG. 11(c) shows a map of the integrated sound speed c̅ₒₚₜ (\hat{x}, t) obtained in a liver imaging experiment. This integrated sound speed map can be used to generate a new ultrasound image in which the axial dimension is no longer determined by the echo time t but by the actual depth of the scatterers.

The result of the method according to this second embodiment is illustrated in FIG. 15. FIG. 15(a) shows the initial ultrasound image in which the axial dimension is an effective depth dictated by the echo time t and the sound speed model c₀.

The first step of the correction process consists of considering, for each pixel (x, t) of the image, the value of the confocal signal obtained for the integrated sound speed model corresponding to c̅ₒₚₜ (\hat{x}, t).

The image obtained in FIG. 15(b) shows much better contrast than the initial image.

The second step of the process consists of repositioning each pixel in depth by axially shifting it by a depth Δz=(c̅ₒₚₜ(x,t)−c₀)t/2. The corresponding displacement map is shown in FIG. 15(a). The image obtained via Equation 26 is now a function of the depth rather than the echo time t. The tissue interfaces show better lateral coherence, particularly at shallow depths where transverse variations in the sound speed are largest and their impact on the quality of the ultrasound image is most significant.

Correction of the axial aberrations of the image is also accompanied by a drastic reduction in transverse aberrations, leading to a significant improvement in the transverse resolution and contrast of the image, as shown in FIG. 16(c).

The integrated sound speed map or image obtained can also be used to estimate a map of the local sound speed, as shown for example in FIG. 11(d).

Thus, the method may comprise a step in which local sound speeds c(r) are calculated for a set of pairs {x, t} (i.e. positions in the medium) from the values of the optimal sound speed models c̅ₒₚₜ obtained for the focal spot images of the pairs {x, t}.

A local sound speed image of the medium can then be formed, and it is possible to display it on the display device 43. This local sound speed image is important for the clinician because, when combined with other information, it is useful for identifying certain pathological conditions or diseases such as hepatic steatosis.

Finally, once a local sound speed field has been acquired, it is then possible to iterate the method described above to obtain a more accurate ultrasonic characterisation of the medium, by recalculating a de-scanned reflection matrix R′ using the local sound speed values determined in a previous step.

We will now describe in greater detail an example of a method for recovering a map or image of the local sound speed c(r) at each point in the image from a map or image of the integrated sound speed. For this purpose, we must invert the following equation:

$$\frac{\overline{z}}{\overline{c} ( \overline{z} )} = {\int }_{0}^{\overline{z}} \frac{dz}{c ( z )}$$

for example by the method developed in the following document:
“Local speed of sound estimation in tissue using pulse-echo ultrasound: Model-based approach.”, Jakovljevic M, et al., Journal of the Acoustical Society of America. 2018 July; 144(1):254.

Under a paraxial approximation, the above equation can be rewritten in the following matrix form:

$$A \times \sum \times \overline{\sum } \qquad ( 27 )$$

Which can be expressed in terms of matrix coefficients as follows:

$$( \begin{matrix}1 & 0 & 0 & 0 & … & 0 \\ \frac{1}{2} & \frac{1}{2} & 0 & 0 & … & 0 \\ \frac{1}{3} & \frac{1}{3} & \frac{1}{3} & 0 & … & 0 \\ \frac{1}{4} & \frac{1}{4} & \frac{1}{4} & \frac{1}{4} & … & 0 \\ … & … & … & … & … & … \\ \frac{1}{N} & \frac{1}{N} & \frac{1}{N} & \frac{1}{N} & … & \frac{1}{N}\end{matrix} ) \times ( \begin{matrix}σ ( x , {z}_{1} ) \\ σ ( x , {z}_{2} ) \\ σ ( x , {z}_{3} ) \\ σ ( x , {z}_{4} ) \\ … \\ σ ( x , {z}_{N} )\end{matrix} ) = ( \begin{matrix}\overline{σ} ( x , {z}_{1} ) \\ \overline{σ} ( x , {z}_{2} ) \\ \overline{σ} ( x , {z}_{3} ) \\ \overline{σ} ( x , {z}_{4} ) \\ … \\ \overline{σ} ( x , {z}_{N} )\end{matrix} ) \qquad ( 29 )$$

With σ(x, z)=c⁻¹(x, z) et σ̅=c̅⁻¹(x, z) presenting the local and integrated slowness maps. The inversion of matrix A leads to the following relationship:

$$\sum = {A}^{- 1} \times \overline{\sum } \qquad ( 28 )$$

Which can be rewritten in terms of matrix coefficients as follows:

$$( \begin{matrix}σ ( x , {z}_{1} ) \\ σ ( x , {z}_{2} ) \\ σ ( x , {z}_{3} ) \\ σ ( x , {z}_{4} ) \\ … \\ σ ( x , {z}_{N} )\end{matrix} ) = ( \begin{matrix}1 & 0 & 0 & 0 & 0 & 0 \\ - \frac{1}{2} & 1 & \frac{1}{2} & 0 & 0 & 0 \\ 0 & - 1 & 1 & 1 & 0 & 0 \\ 0 & 0 & - \frac{3}{2} & 1 & \frac{3}{2} & 0 \\ … & … & … & … & … & … \\ 0 & 0 & 0 & 0 & - \frac{N - 1}{2} & 1\end{matrix} ) \times ( \begin{matrix}\overline{σ} ( x , {z}_{1} ) \\ \overline{σ} ( x , {z}_{2} ) \\ \overline{σ} ( x , {z}_{3} ) \\ \overline{σ} ( x , {z}_{4} ) \\ … \\ \overline{σ} ( x , {z}_{N} )\end{matrix} ) \qquad ( 29 )$$

The inversion of the integrated sound speed map c̅ₒₚₜ (x, t) shown in FIG. 11(c) provides an estimator cₒₚₜ (x, t) of the local sound speed map c(x, t) illustrated in FIG. 11(d). This local sound speed map reveals the different tissue layers present in the liver imaging experiment, particularly the fat layer and the muscle tissues, which present strongly contrasting sound speeds. Accurate and reliable measurement of the speed of sound in the liver is crucial for detecting diseases such as hepatic steatosis. The speed of sound measured here in the patient's liver is particularly low: <c>=1450 m/s whereas the speed of sound in a healthy liver is typically around 1600 m/s. The patient in question does indeed suffer from hepatic steatosis, which was confirmed by other examination methods that are more costly, time-consuming and/or invasive. This disease could ultimately be detected by a simple ultrasound measurement of the speed of sound in the liver as disclosed herein.

It should be noted that the inversion method mentioned above relies on a very strong approximation in which the integrated sound speed at a point c̅(x, z) depends only on the value of the local sound speed directly beneath it c(x, z′<z).

Other, more sophisticated methods can take into account oblique paths and the refraction phenomena to which the incident and reflected waves are subjected. They can be used to invert the integrated sound speed map obtained with the present method.

The local sound speed map can be used to construct a propagation model that is more advanced than the initial speed model in order to obtain a more accurate estimate of the reflectivity of the medium via an ultrasound image obtained using delay laws based on the sound speed map.

Similarly, the entire method described above for a uniform sound speed model c₀ can also be used to optimise a more complex speed model c₀(r) using more sophisticated optimisation algorithms aimed at maximising the coherent confocal signal and/or the Gouy phase gradient.

1-20. (canceled)

21. A method for ultrasonic characterisation of a medium comprising:
a step of generating a series of incident ultrasonic waves (USᵢₙ) in a region of said medium, by means of an array (10) of transducers (11), said series of incident ultrasonic waves forming a transmission basis (i); and
a step of generating an experimental reflection matrix Rᵤᵢ(t) defined between the transmission basis (i) at the input and a reception basis (u) at the output, the coefficients of this matrix corresponding to the signals received by the transducers induced by the reflected ultrasonic waves;
a step of determining a de-scanned reflection matrix R′,
which comprises, for a set of points of the medium of abscissa x and echo time t,
responses of the medium calculated by beamforming according to a sound speed model c₀ from the experimental reflection matrix Rᵤᵢ(t), between an input virtual transducer of spatial position rᵢₙ=(x, zₜ) and an output virtual transducer of spatial position rₒᵤₜ=(x+Δx, zₜ), both virtual transducers being at the same depth zₜ, zₜ being the depth of the isochronous volume expected for the sound speed c₀, the isochronous volume being the set of points of the medium contributing to the signals received at time t,
the responses of the de-scanned reflection matrix R′ being determined for a set of lateral offset values Δx including at least Δx=0 and for a set of sound speed models c₀,
said de-scanned reflection matrix being formed such that each row corresponds to an element of a pair {Δx, c₀} which corresponds to a focal spot image, and each column corresponds to an element of a pair {x, t}, which is written as:
$${R}^{′} = [ {R}^{′} ( \{ Δ x , {c}_{0} \} , \{ x , t \} ) ] ,$$
and
an extraction step in which, from each focal spot image, an optimal lateral offset Δxₒₚₜ and an optimal sound speed model c̅ₒₚₜ, close to the maximum in said focal spot image, are determined.

22. The method according to claim 21, further comprising:
a step of forming the ultrasound image by computing the reflectivity at a plurality of points in the medium, from the de-scanned reflection matrix R′, the reflectivity of each of these points in the medium corresponding to the response of the medium between the input virtual transducer at spatial position rᵢₙ=(x, zₜ) and the output virtual transducer at spatial position rₒᵤₜ=(x+Δxₒₚₜ, zₜ), zₜ being the expected depth of the isochronous volume for the optimal sound speed model c̅ₒₚₜ and said reflectivity being defined by:
$${I}_{2} ( x , t ) = \vert {R}^{′} ( \{ Δ x = Δ {x}_{opt} ( x , t ) , {c}_{0} = {\overline{c}}_{opt} ( x , t ) \} , \{ x , t \} ) \vert .$$

23. The method according to claim 22, in which the ultrasound image is transformed in the depth dimension, using the optimal sound speed model c̅ₒₚₜ, by:
$${\overline{I}}_{2} ( x , \overline{z} ) = {I}_{2} ( x , t = 2 \overline{z} / {\overline{c}}_{opt} ( x , t ) ) ,$$
in which \tilde{z} is the estimate of the depth z of a pixel of the ultrasound image.

24. The method according to claim 22, in which the ultrasound image is displayed on a display device.

25. The method according to claim 21, in which:
local sound speeds c(r) are calculated, for a set of pairs {x, t}, from the optimal sound speed values c̅ₒₚₜ obtained for the focal spot images of said pairs {x, t}.

26. The method according to claim 25, further comprising a step of forming a local sound speed image of the medium from said set of local sound speed values, said local sound speed image being displayed on a display device.

27. The method according to claim 25, in which a de-scanned reflection matrix R′ is recalculated using the local sound speed values.

28. The method according to claim 21, further comprising, prior to the extraction step:
a step of spatio-temporal averaging of the de-scanned reflection matrix R′ in which the values of the de-scanned reflection matrix are averaged locally, along the lateral dimension x and the echo time t, for each element of a pair {Δx, c₀}, the focal spot images then being averaged.

29. The method according to claim 28, in which:
in the spatio-temporal averaging step, an incoherent point spread function PSF_{inc} is computed by the following formula:
$${PSF}_{inc} ( \{ Δ x , {c}_{0} \} , \{ x , t \} ) = \sqrt{{〈 {\vert {R}^{′} ( \{ Δ x , {c}_{0} \} , \{ {x}^{′} , {t}^{′} \} ) W ( {x}^{′} - x , {t}^{′} - t ) \vert}^{2} 〉}_{[ {x}^{′} , {t}^{′} ]}}$$
for a set of lateral offset values Δx including at least Δx=0 and for a set of sound speed model values c₀, in order to obtain the focal spot image for the lateral position x and the echo time t,
in which:
<.> is an averaging operator with respect to the parameters of the pair {x′, t′}
R′ is the de-scanned reflection matrix,
W(x′, t′) is a spatio-temporal weighting window using the same parameters, and
in the extraction step, a maximum point of the focal spot image is sought, corresponding to a maximum of the magnitude of the points of the focal spot image, and the optimal sound speed model c̅ₒₚₜ is the ordinate of said maximum point in said focal spot image.

30. The method according to claim 28, in which:
in the spatio-temporal averaging step, a singular value decomposition (SVD) of a local focal spot matrix R^{(l)} is computed, said local focal spot matrix being derived from the de-scanned reflection matrix R′ by:
$${R}^{( l )} ( \{ x , t \} ) = [ {R}^{′} ( \{ Δ x , {c}_{0} \} , \{ {x}^{′} , {t}^{′} \} W ( {x}^{′} - * x , {t}^{′} - t ) ]$$
in which:
R′ is the de-scanned reflection matrix,
W(x′, t′) is a spatio-temporal weighting window using the same parameters.
The singular value decomposition (SVD) of said local matrix is written as:
$${R}^{( l )} ( x , t ) = {\sum }_{p} {λ}_{p} {U}_{p} ( \{ x , t \} ) {V}_{p}^{†} ( \{ x , t \} )$$
in which:
λₚ are the singular values of said local matrix,
Uₚ are the output eigenvectors,
Vₚ are the input eigenvectors,
a coherent point spread function PSF_{coh} is computed by the following formula:
$${PSF}_{coh} ( \{ Δ x , {c}_{0} \} , \{ x , t \} ) = {U}_{1} ( \{ Δ x , {c}_{0} \} , \{ x , t \} )$$
for a set of lateral offset values Δx including at least Δx=0 and for a set of sound speed model values c₀, in order to obtain the focal spot image for the lateral position x and the echo time t, and
in the extraction step, a maximum point of the focal spot image is sought, corresponding to:
a maximum of the magnitude of the points of the focal spot image, or
a maximum of the magnitude of the derivative, with respect to the sound speed c₀, of the points of the focal spot image, or
a maximum of the magnitude of the derivative, with respect to the sound speed c₀, of the imaginary part of the points of the focal spot image, and
the optimal sound speed model c̅ₒₚₜ is the ordinate of said maximum point in said focal spot image.

31. The method according to claim 21, in which, in the extraction step, the lateral offset Δxₒₚₜ is considered to be zero, Δxₒₚₜ=0.

32. The method according to claim 21, in which, in the step of determining a de-scanned reflection matrix R′, the de-scanned reflection matrix R′ is formed only for lateral offset values Δx equal to zero, Δx=0.

33. The method according to claim 28, in which, in the spatio-temporal averaging step, the values of the de-scanned reflection matrix R′ are averaged to determine a focal spot image, for each element of a pair {Δx=0, Δz}, along the lateral dimension x and the echo time t.

34. The method according to claim 21, in which, in the extraction step, the optimal sound speed model c̅ₒₚₜ is determined from the points of the focal spot image having a lateral offset Δx equal to zero, Δx=0.

35. The method according to claim 21, in which the beamforming calculations between the input virtual transducer and the output virtual transducer are performed based on the experimental reflection matrix Rᵤᵢ(t), using a forward delay of the ultrasonic waves between the transmission basis (i) and the input virtual transducer, and using a return delay of the ultrasonic waves between the output virtual transducer and the transducers of the reception basis (u).

36. The method according to claim 21, in which the de-scanned reflection matrix R′ is calculated from the experimental reflection matrix Rᵤᵢ by the following formula:
$${R}^{′} ( \{ Δ x , {c}_{0} \} , \{ {x}^{′} , {t}^{′} \} = \frac{1}{{N}_{in} {N}_{out}} {\sum }_{{i}_{iu} , {u}_{out}} {R}_{ui} ( {u}_{out} , {i}_{in} , t + {τ}^{′} ( x , {i}_{in} , {z}_{0} , {c}_{0} ) + τ ( x + Δ x , {u}_{out} , {z}_{t} , {c}_{0} ) )$$
in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, uᵢₙ, t) is the element of the experimental reflection matrix Rᵤᵢ(t) recorded by the transducer at spatial position Wout following the transmission of index iᵢₙ in the transmission basis (i), at time t,
c₀ being the expected speed of sound.

37. The method according to claim 21, in which the de-scanned reflection matrix R′ is computed from a focused reflection matrix R by the following formula:
$${R}^{′} ( \{ Δ x , {c}_{0} \} , \{ x , t \} ) = R ( x + Δ x , x , t , {c}_{0} )$$
in which:
the de-scanned reflection matrix R′ is formed such that each row is composed of elements of a pair {Δx, Δz} and each column is composed of elements of a pair {x, t}, and
the focused reflection matrix R is calculated from the experimental reflection matrix Rᵤᵢ(t) by the following formula:
$$R ( {x}_{out} , {x}_{in} , t , {c}_{0} ) = \frac{1}{{N}_{in} {N}_{out}} {\sum }_{{i}_{in} , {u}_{out}} {R}_{ui} ( {u}_{out} , {i}_{in} , t + {τ}^{′} ( {x}_{in} , {i}_{in} , {z}_{t} , {c}_{0} ) + τ ( {x}_{out} , {u}_{out , {z}_{t} , {c}_{0}} ) )$$
in which:
Nᵢₙ is the number of elements in the transmission basis (i)
Nₒᵤₜ is the number of elements in the reception basis (u),
Rᵤᵢ(t) is the experimental reflection matrix, of which
Rᵤᵢ(uₒᵤₜ, iᵢₙ, t) is the element of the experimental reflection matrix Rᵤᵢ(t) recorded by the transducer at spatial position uₒᵤₜ following the transmission of index iᵢₙ in the transmission basis (i) and at time t,
c₀ being the expected speed of sound.

38. The method according to claim 21, in which a video is formed from a sequence of ultrasound images produced over a first number N of iterations of the steps of the method.

39. The method according to claim 38, in which, among the N iterations, a second number P, strictly smaller than N, of iterations do not include the steps of determining the de-scanned reflection matrix R′, the spatio-temporal averaging steps, or the steps of extracting the optimal sound speed model, and for these P iterations, in the ultrasound image formation step, the ultrasound image is obtained from an optimal sound speed extracted during a previous iteration.

40. A system for ultrasonic characterisation of a medium, the system comprising:
an array of transducers adapted to generate a series of incident ultrasonic waves in a region of the medium, and to record, as a function of time, the ultrasonic waves backscattered by said region; and
a computing unit linked to the transducer array and adapted to implement the method according to claim 21.
